(19) United States
(12) Patent Application Publication
(10) Pub. No.: US 2005/0064435 A1
(43) Pub. Date: Mar. 24, 2005

(54) Programmable molecular barcodes

(76) Inventors: Xing Su, Cupertino, CA (US); Tae-Woong Koo, South San Francisco, CA (US); Andrew A. Berlin, San Jose, CA (US); Lei Sun, Santa Clara, CA (US); Narayanan Sundararajan, San Francisco, CA (US); Mineo Yamakawa, Campbell, CA (US)

Correspondence Address: LISA A. HAILE, Ph.D.;ATTORNEY FOR INTEL CORPORATION, GRAY CARY WARE & FREIDENRICH LLP, 4365 Executive Drive, Suite 1100, San Diego, CA, 92121-2133, US

(21) Appl. No.: 10/670,701

(22) Filed: Sep. 24, 2003

(51) Int. Cl. C12Q 1/68; G01N 33/53; G06K 7/10

(52) U.S. Cl. 435/6; 435/7.1; 235/462.1

(57) ABSTRACT

The present disclosure concerns methods for producing and/or using molecular barcodes. In certain embodiments of the invention, the barcodes comprise polymer backbones that may contain one or more branch structures. Tags may be attached to the backbone and/or branch structures. The barcode may also comprise a probe that can bind to a target, such as proteins, nucleic acids and other biomolecules or aggregates. Different barcodes may be distinguished by the type and location of the tags. In other embodiments, barcodes may be produced by hybridization of one or more tagged oligonucleotides to a template, comprising a container section and a probe section. The tagged oligonucleotides may be designed as modular code sections, to form different barcodes specific for different targets. In alternative embodiments, barcodes may be prepared by polymerization of monomeric units. Bound barcodes may be detected by various imaging modalities, such as, surface plasmon resonance, fluorescent or Raman spectroscopy.

### FIELD

The present methods, compositions and apparatus relate to the field of molecular barcodes. Particular embodiments of the invention concern methods for creating molecular barcodes from organic polymer backbones. Multiple molecular barcodes may be produced using the same backbone by attaching tags to different sites on the backbone. In other embodiments, molecular barcodes may include a probe region and one or more code components. In other embodiments, molecular barcodes may include polymeric Raman labels attached to one or more probes for detection of target molecules.

### BACKGROUND

Detection and/or identification of biomolecules are of use for a variety of applications in medical diagnostics, forensics, toxicology, pathology, biological warfare, public health and numerous other fields. Although the principle classes of biomolecules studied are nucleic acids and proteins, other biomolecules such as carbohydrates, lipids, polysaccharides, lipids, fatty acids and others are of interest. A need exists for rapid, reliable and cost effective methods of identification of biomolecules, methods of distinguishing between similar biomolecules and analysis of macromolecular complexes such as pathogenic spores or microorganisms.

Standard methods for nucleic acid detection, such as Southern blotting, Northern blotting or binding to nucleic acid chips, rely on hybridization of a fluorescent, chemiluminescent or radioactive probe molecule with a target nucleic acid molecule. In oligonucleotide hybridization-based assays, a labeled oligonucleotide probe that is complementary in sequence to a target nucleic acid is used to bind to and detect the nucleic acid. More recently, DNA (deoxyribonucleic acid) chips have been designed that can contain hundreds or thousands of attached oligonucleotide probes for binding to target nucleic acids. Problems with sensitivity and/or specificity may result from nucleic acid hybridization between sequences that are not completely complementary. Alternatively, the presence of low levels of a target nucleic acid in a sample may not be detected.

A variety of techniques are available for identification of proteins, polypeptides and peptides. Commonly, these involve binding and detection of antibodies. Although antibody-based identification is fairly rapid, such assays may occasionally show high levels of false positives or false negatives. The cost of these assays is high and simultaneous assaying of more than one target is difficult. Further, the methods require that an antibody be prepared against the target protein of interest before an assay can be performed.

A number of applications in molecular biology, genetics, disease diagnosis and prediction of drug responsiveness involve identification of nucleic acid sequence variants. Existing methods for nucleic acid sequencing, including Sanger dideoxy sequencing and sequencing by hybridization, tend to be relatively slow, expensive, labor intensive and may involve use of radioactive tags or other toxic chemicals. Existing methods are also limited as to the amount of sequence information that may be obtained in one reaction, typically to about 1000 bases or less. A need exists for more rapid, cost-effective and automated methods of nucleic acid sequencing.

### BRIEF DESCRIPTION OF THE DRAWINGS

The following drawings form part of the present specification and are included to further demonstrate certain aspects of the disclosed embodiments of the invention. The embodiments may be better understood by reference to one or more of these drawings in combination with the detailed description presented herein.

FIG. 1. illustrates an exemplary method for generating a barcode 100 with an organic backbone 110 modified with branches 120 and tags 130. The barcode 100 may include a probe moiety 150 to bind to a target. The tags 130 may be subject to additional modification, for example by binding to an antibody 140.

FIG. 2 illustrates an exemplary method for generating different barcodes 201, 202, 203 utilizing the same backbone. Tags 240, 250, 260 may be placed in different locations to generate distinguishable barcodes 201, 202, 203. Binding of the barcode 201, 202, 203 to targets may be mediated by probe moieties 210, 220, 230 attached to the barcodes 201, 202, 203.

FIG. 3 illustrates an example of several barcodes 301, 302, 303, 304 with single stranded nucleic acid backbones. Tags 310, 320, 330 are added at various sites on the backbone to generate different spectra that may be identified, for example, by Raman spectroscopy. Barcodes with the same tag 330 attached at different sites on the barcode 302, 303, 304 may generate distinguishable Raman spectra.

FIG. 4 illustrates an example of Raman spectra generated by the barcodes disclosed in FIG. 3. Barcodes 301, 302, 303, and 304 are represented in the graph.

FIG. 5 illustrates an exemplary method for generating a barcode using a variety of short oligonucleotides 520 of known sequence attached to one or more tags 510. The oligonucleotide-tag molecules may be assembled into a barcode by hybridization to a template molecule 500. The template 500 may comprise a container section 540 for oligonucleotide-tag hybridization and a probe section 550 for binding to a target molecule, such as a nucleic acid. In alternative embodiments, the probe 550 may comprise, for example, an aptamer sequence that can bind to proteins, peptides or other types of targets.

FIG. 6 represents a schematic of an exemplary method for making barcodes, including creating code components 601 602 603 604 by attaching a tag moiety to an oligonucleotide or nucleic acid, creating a template 606 and hybridizing the code components to the template 605 to generate a barcode 607.

FIG. 7 represents a schematic of an exemplary method for utilizing a barcode generated by the method of FIG. 6 to identify the presence or absence of a complementary target strand.

FIG. 8 represents an example of a plot of SERS (surface enhanced Raman spectroscopy) spectra produced by several Raman tags 801 802 803 804 805 806.

FIG. 9 illustrates an example of a polymeric Raman label 910. Monomeric units 901,902 are linked by a covalent bond 906 generated from the interaction of a functional group 904, 908 attached to a backbone 909 with another functional group 904, 908 on the end of the growing polymeric chain. Optionally, additional units 903 may be added.

FIG. 10 represents a schematic of an exemplary method for generating a polymeric Raman label. A solid support 1001 is used to attach a component 1005 (e.g. a portion of the polymeric Raman label). The open end 1104 of the component 1005 is de-protected and a monomeric unit 1010 is attached to the component 1005 via a deprotected functional group 1006 of the monomeric unit 1010. Raman tags 1002, 1003, 1008 are attached to the polymeric Raman label.

FIG. 11A represents another exemplary method for generating polymeric Raman labels 1105. A first reaction is used to attach functional groups 1102a, 1102b to Raman tags 1101a, 1101b, generating functionalized Raman tags 1103a, 1103b. A second reaction is used to polymerize functionalized Raman tags 1103a, 1103b to form sub-polymeric Raman labels 1104a, 1104b. Each sub-polymeric Raman label 1104a, 1104b comprises a predetermined number of monomeric Raman tags 1103a, 1103b. In this example, a first sub-polymer 1104a comprises “n” copies of a first monomer 1103a and a second sub-polymer 1104b comprises “m” copies of a second monomer 1103b. A predetermined ratio of the sub-polymeric Raman labels 1104a, 1104b may be mixed and cross-linked to form a polymeric Raman label 1105.

FIG. 11B represents yet another exemplary method for generating polymeric Raman labels. A polymer molecule 1109 with functional groups 1112 may be combined with different Raman tags 1110 to form a polymeric Raman label 1111. The number of each type of Raman tag 1110 may be predetermined to produce a polymeric Raman label 1111 with specified spectroscopic properties.

FIG. 12 illustrates several examples of polymeric Raman labels linked to one or more probes 1206 to identify a target molecule. The first example 1201 shows a polymeric Raman label 1204 attached to a probe 1206 via a linker 1205. The second example 1202 shows two polymeric Raman labels 1204 linked 1205 to a nanoparticle 1207 and additional linkers 1205 attaching the nanoparticle 1207 to two probes 1206. The third example 1203 shows multiple probes 1206 attached via linkers 1205 to a nanoparticle and multiple Raman tags 1208 attached to the nanoparticle 1207.

FIG. 13 represents an example of a plot of SERS (surface enhanced Raman spectroscopy) spectra produced by several Raman tags of a modified nucleic acid, adenine.

### DESCRIPTION OF ILLUSTRATIVE EMBODIMENTS

The following detailed description contains numerous specific details in order to provide a more thorough understanding of the disclosed embodiments of the invention. However, it will be apparent to those skilled in the art that the embodiments may be practiced without these specific details. In other instances, devices, methods, procedures, and individual components that are well known in the art have not been described in detail herein.

Definitions

As used herein, “a” or “an” may mean one or more than one of an item.

As used herein, a “multiplicity” of an item means two or more of the item.

As used herein, “nucleic acid” encompasses DNA, RNA (ribonucleic acid), single-stranded, double-stranded or triple stranded and any chemical modifications thereof. Virtually any modification of the nucleic acid is contemplated. A “nucleic acid” may be of almost any length, from oligonucleotides of 2 or more bases up to a full-length chromosomal DNA molecule. Nucleic acids include, but are not limited to, oligonucleotides and polynucleotides.

A “probe” molecule is any molecule that exhibits selective and/or specific binding to one or more targets. In various embodiments of the invention, each different probe molecule may be attached to a distinguishable barcode so that binding of a particular probe from a population of different probes may be detected. The embodiments are not limited as to the type of probe molecules that may be used. Any probe molecule known in the art, including but not limited to oligonucleotides, nucleic acids, antibodies, antibody fragments, binding proteins, receptor proteins, peptides, lectins, substrates, inhibitors, activators, ligands, hormones, cytokines, etc. may be used. In certain embodiments, probes may comprise antibodies, aptamers, oligonucleotides and/or nucleic acids that have been covalently or non-covalently attached to one or more barcodes to identify different targets.

Illustrative Embodiments

The disclosed methods, compositions and apparatus are of use for detection, identification and/or tagging of biomolecules, such as nucleic acids and proteins. In particular embodiments of the invention, the methods, compositions and apparatus may be used to generate multiple barcodes from a single organic backbone by making various modifications of the backbone. The embodiments are not limited to a single backbone, but may utilize one or more different backbones. Advantages include the ability to generate different barcodes with the same backbone by varying the attachment sites of tags along the backbone. Other embodiments concern generating polymeric Raman labels for rapid identification of or for tagging biomolecules. Other advantages include the sensitive and accurate detection and/or identification of polypeptides.

Barcodes by Synthesis

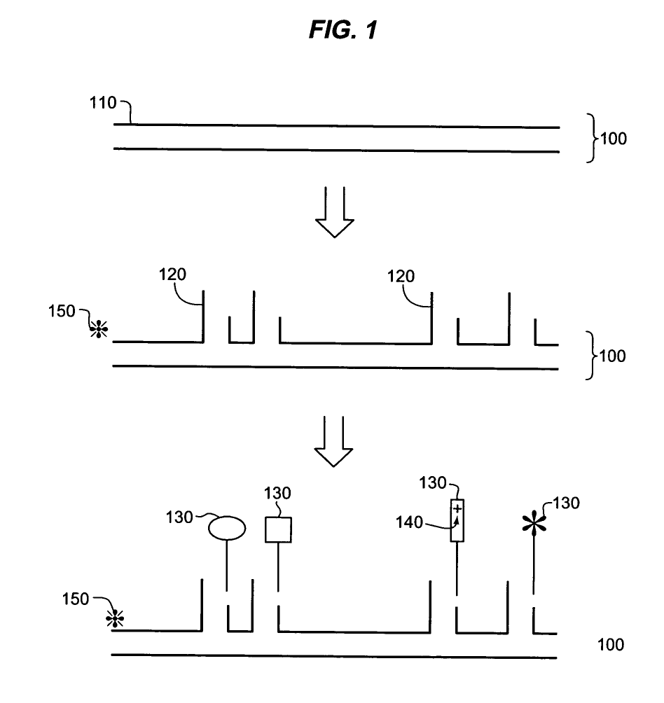

In one embodiment of the invention, illustrated in FIG. 1, barcode backbones 110 may be formed from polymer chains comprising organic structures, including any combination of nucleic acid, peptide, polysaccharide, and/or chemically derived polymer sequences. In certain embodiments, the backbone 110 may comprise single or double-stranded nucleic acids. In some embodiments, the backbone may be attached to a probe moiety 150, such as an oligonucleotide, antibody or aptamer. The backbone 110 may be modified with one or more branch structures 120 to create additional morphological diversity and tag attachment sites. Branch structures 120 may be formed using techniques well known in the art. For example, where the barcode 100 comprises a double-stranded nucleic acid, branch structures 120 may be formed by synthesis of oligonucleotides and hybridization to a single-stranded template nucleic acid. The oligonucleotides may be designed so that part of the sequence (e.g., the 5′ end) is complementary to the template and part (e.g., the 3′ end) is not. Thus, the barcode 100 will contain segments of double-stranded sequence and short segments of single-stranded branch structures 120. As disclosed in FIG. 1, tags 130 may be added to the barcode, for example by hybridization of labeled 130 oligonucleotides that are complementary in sequence to the single-stranded portions of the branch structures 120.

Oligonucleotide mimetics may be used to generate the organic backbone 110. Both the sugar and the internucleoside linkage, i.e., the backbone, of the nucleotide units may be replaced with novel groups. The probes 150 may be used to hybridize with an appropriate nucleic acid target compound. One example of an oligomeric compound or an oligonucleotide mimetic that has been shown to have excellent hybridization properties is referred to as a peptide nucleic acid (PNA). In PNA compounds, the sugar-backbone of an oligonucleotide is replaced with an amide containing backbone, for example an aminoethylglycine backbone. In this example, the nucleobases are retained and bound directly or indirectly to an aza nitrogen atom of the amide portion of the backbone. Several United States patents that disclose the preparation of PNA compounds include, for example, U.S. Pat. Nos. 5,539,082; 5,714,331; and 5,719,262. In addition, PNA compounds are disclosed in Nielsen et al. (Science, 1991, 254, 1497-15).

In order to distinguish one barcode 100 from another, tags 130 may be added directly to the backbone 110 or to one or more branch structures 120. Barcodes 100 may be further modified by attaching another molecule 140 (for example an antibody) to one or more of the tags 130. Where bulky groups are used, modification of tag moieties 130 attached to branch sites 120 would provide lower steric hindrance for probe 150 interactions with target molecules. The tags 130 may be read by an imaging modality, for example fluorescent microscopy, FTIR (Fourier transform infra-red) spectroscopy, Raman spectroscopy, electron microscopy, and surface plasmon resonance. Different variants of imaging are known to detect morphological, topographic, chemical and/or electrical properties of tags 130, including but not limited to conductivity, tunneling current, capacitive current, etc. The imaging modality used will depend on the nature of the tag moieties 130 and the resulting signal produced. Different types of known tags 130, including but not limited to fluorescent, Raman, nanoparticle, nanotube, fullerenes and quantum dot tags 130 may be used to identify barcodes 100 by their topographical, chemical, optical and/or electrical properties. Such properties will vary as a function both of the type of tag moiety 130 used and the relative positions of the tags 130 on the backbone 110 or branch structures 120, resulting in distinguishable signals generated for each barcode 100.

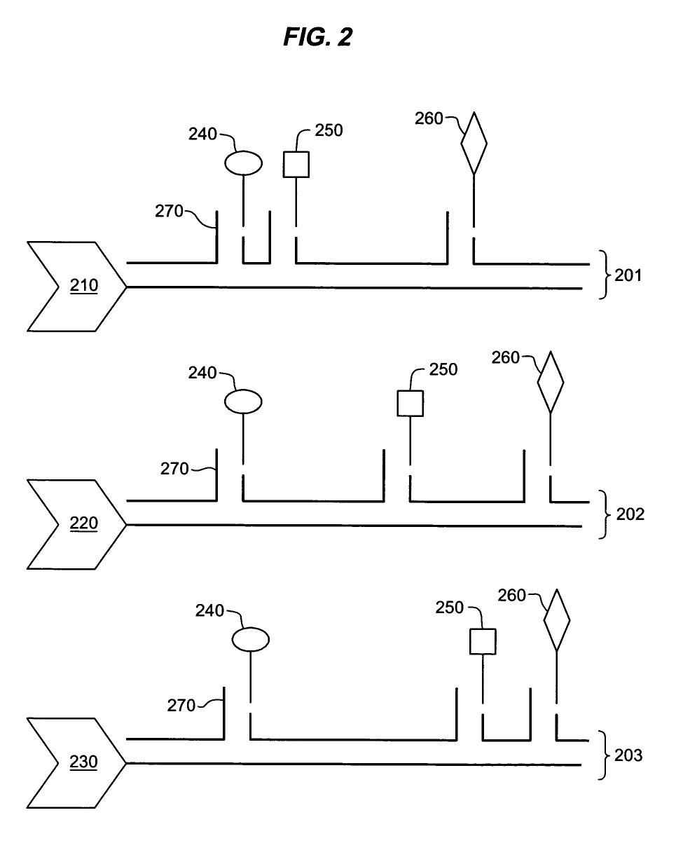

As shown in FIG. 2, different probes 210, 220, 230 that recognize specific targets may be attached to distinguishable barcodes 201, 202, 203. In this exemplary embodiment, multiple tags 240, 250, 260 may be attached to barcodes 201, 202, 203 at different sites. The tags 240, 250, 260 may comprise, for example, Raman tagss or fluorescent tags. Because adjacent tags may interact with each other, for example by fluorescent resonance energy transfer (FRET) or other mechanisms, the signals obtained from the same set of tag moieties 240, 250, 260 may vary depending upon the locations and distances between the tags 240, 250, 260 (see Example 1). Thus, barcodes 201, 202, 203 with similar or identical backbones may be distinguishably labeled. Specificity of target molecule binding may be provided by attachment of probes 210, 220, 230, such as antibodies, aptamers or oligonucleotides, to the barcodes 201, 202, 203. Because the barcode 201, 202, 203 signal corresponding to a given probe 210, 220, 230 specificity is known, it is possible to analyze complex mixtures of molecules and to detect individual species by determining which probes 210, 220, 230 bind to targets in the sample.

In certain embodiments of the invention, illustrated in FIG. 1 and FIG. 2, the backbone 110 of a barcode 100, 201, 202, 203 may be formed of phosphodiester bonds, peptide bonds, and/or glycosidic bonds. For example, standard phosphoramidite chemistry may be used to make backbones 110 comprising DNA chains. Other methods for making phosphodiester linked backbones 110 are known, such as polymerase chain reaction (PCR™) amplification. The ends of the backbone 110 may have different functional groups, for example, biotins, amino groups, aldehyde groups or thiol groups. The functional groups may be used to bind to probe moieties 150, 210, 220, 230 or for attachment of tags 130, 240, 250, 260. Tags 130, 240, 250, 260 maybe further modified to obtain different sizes, electrical or chemical properties to facilitate detection. For example, an antibody could be used to bind to a digoxigenin or a fluorescein tag 130, 240, 250, 260. Streptavidin could be used to bind to biotin tags 130, 240, 250, 260. Metal atoms may be deposited on the barcode 100, 201, 202, 203 structure, for example by catalyzed reduction of a metal ion solution using an enzyme tag 130, 240, 250, 260. Where the barcode 100, 201, 202, 203 comprises a peptide moiety, the peptide may be phosphorylated for tag 130, 240, 250, 260 modification 140. Modified 140 tags 130, 240, 250, 260 may be detected by a variety of techniques known in the art.

In certain embodiments of the invention, solutions containing one or more barcodes 100, 201, 202, 203 may be applied to objects for security tracking purposes. Such methods are known in the art. For example, a British company (SmartWater Ltd.) has developed methods to mark valuables with fluids containing strands of digital DNA. The DNA is virtually impossible to wash off of the article and may be used to uniquely identify expensive items or heirlooms. The DNA may be detected by any forensic laboratory. Such methods may also be utilized to mark items with the molecular barcodes 100, 201, 202, 203 disclosed herein. In such applications, detection of the barcode 100, 201, 202, 203 would not require forensic analysis based on DNA sequence.

Barcodes by Hybridization

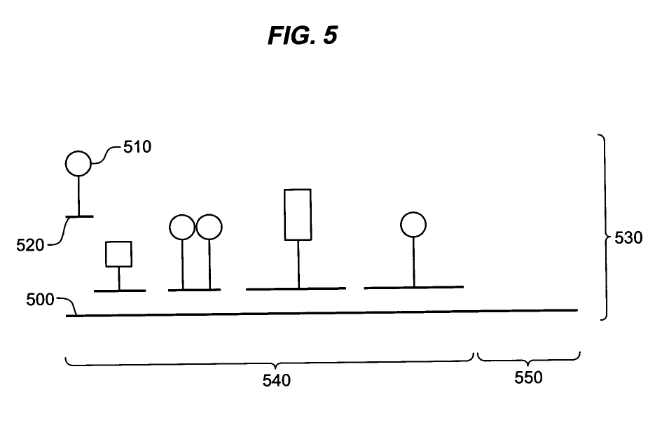

Other embodiments of the invention, illustrated in FIG. 5, concern methods for generating barcodes 530 by hybridization. In this embodiment, the barcodes 530 comprise nucleic acids 500 hybridized to oligonucleotides 520. One or more tag moieties 510 may be attached to an oligonucleotide 520 of known sequence produced, for example by known chemical synthesis techniques. Various methods for producing tagged oligonucleotides 520 are well known in the art. The barcode 530 is formed by hybridization of a series of tagged oligonucleotides 520 to a single-stranded DNA template 500. The template 500 comprises a container section 540 and a probe section 550. The probe section 550 is designed to hybridize to a complementary target nucleic acid sequence. Alternatively, the probe section 550 may comprise an aptamer sequence that can bind to proteins, peptides or other target biomolecules. In various embodiments, the probe region 540 may between 2 to 30, 4 to 20 or 14 to 15 nucleotides long. The probe 550 length is not limiting and probe sections 550 of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 200, 250 nucleotides or even longer are contemplated.

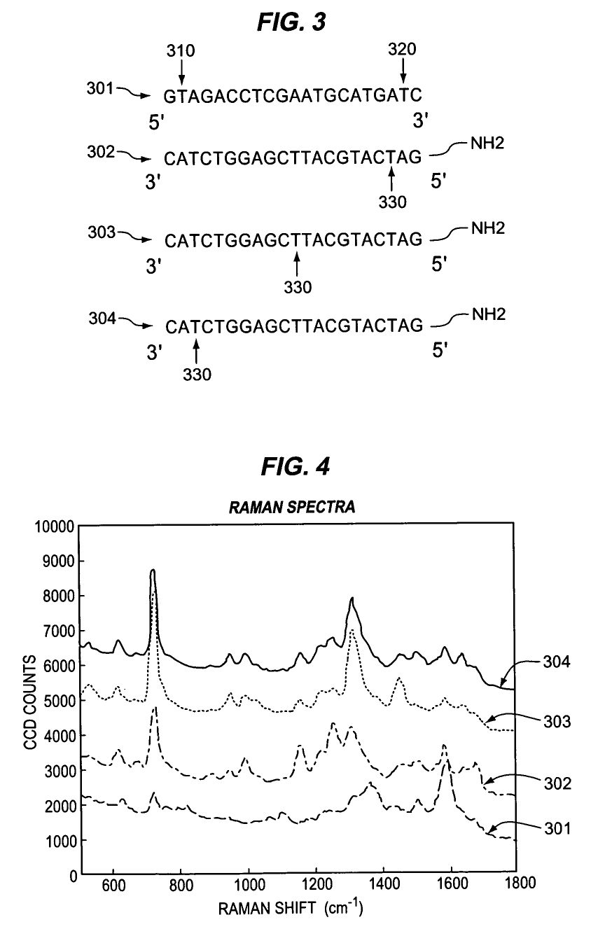

FIG. 3 illustrates exemplary Raman tagged oligonucleotides of use in various embodiments of the invention. The Raman tags 310 320 330 may be attached to different nucleotides of the same oligonucleotide sequence to generate different spectra (FIG. 4). For example, oligonucleotides 302 303 and 304 illustrate the same oligonucleotide sequence where the position of the tag 330 is changed. As shown in, FIG. 4 the Raman spectra for the tagged oligonucleotides 301, 302, 303, 304 disclosed in FIG. 3 are distinguishable. FIG. 4 demonstrates that a small change in position of the same Raman tag 330 attached to the same oligonucleotide sequence 302, 303, 304 may generate different patterns of Raman spectra. (, for more detail, see Examples 1 and 2 below.)

In embodiments of the invention illustrated in FIG. 5, a barcode 530 may be formed when one or more tagged oligonucleotides 520 are allowed to hybridize to a container section 540 of a template molecule 500. The sequences of the tagged oligonucleotides 520 are designed to be complementary to the container section 540, not to the probe section 550. The combination of tag moieties 510 bound by hybridization to the template 500 is selected to provide a distinguishable signal. There is no limitation on the type of signal that may be used and any known detection technique, including but not limited to Raman spectroscopy, FTIR, surface plasmon resonance may be utilized. Following hybridization, the barcode 530 may be separated from unhybridized oligonucleotides 520 and template strands 500 by known methods, including but not limited to ultrafiltration, HPLC (high performance liquid chromatography), hydroxylapatite column chromatography, ultracentrifugation, etc. This method for barcode 530 production has high labeling efficiency and requires a reduced number of tagged oligonucleotides 520 to be produced versus standard techniques, wherein each tagged oligonucleotide 520 comprises a separate and identifiable barcode 530. As will be apparent to the skilled artisan, the method illustrated in FIG. 5 illustrates a combinatorial method for barcode 530 production, allowing formation of a large number of distinguishable barcodes 530 using a much smaller number of tagged oligonucleotides 520.

In certain embodiments of the invention illustrated in FIG. 5, the length of the template 500 sequence may be determined from the sizes of the probe section 550 and the tagged oligonucleotides 520 to hybridize to the container section 540. For example, for a probe section 550 of “n” bases in length and individual tagged oligonucleotides 520 of “m” bases in length, the length of the template 500 is equal to (1+m) times n (or alternatively, (n times m)+n). For example, given a probe section 550 of 9 bases in length and tagged oligonucleotides 520 of 5 bases in length, the length of the template 500 needed to provide unique barcodes for all possible 9-mer probe sequences would be (1+5) times 9, or 54 bases.

Allowing for partial sequence overlap, a given 54 base template may contain up to 50 different 5-mer sequences assuming full hybridization (i.e., a 5-mer can't bind only to the last 4 bases of the template 500). The number of possible different m-mers contained in such a template may also be calculated as equal to (n+(n times m)−m+1). On the other hand, there 4⁵ (or 1024) possible sequences of 5-mer that could be synthesized, since each position of the 5-mer may contain one of four possible bases, and there are five positions. This means that there are 4^{m}−(n+(n times m)−m+1) types of 5-mer that could be used as code components. In the present instance, there are 974 (1024-50) types of 5-mer that could be used as code components. The container section 540 will be designed to hybridize to a series of unique 5-mers, out of the 974 types available. Tagged oligonucleotides 520 comprising the appropriate code sequences may be introduced and hybridized to the container section 540. Each tagged oligonucleotide 520 will contain tags providing a unique signal, so that it may be identified from other code components.

The principle may be illustrated by reference to an exemplary illustration. Where the probe section 550 is 4 bases long (n=4) and the tagged oligonucleotides 520 comprise 3 base sequences (m=3), then the template 500 length will be 16 bases long ((1+3) times 4). This results in a 12 base container section 540 and a 4 base (4-mer) probe section 550. Since m=3, there are 64 (43) possible 3-mer sequences available. Each 16 base template 500 can contain up to fourteen types of 3-mer (4+(3*4)−3+1=14). An arbitrary template 500 sequence is shown in SEQ ID NO:1 below, with the probe section 550 (underlined) to the left and the container section 540 to the right.

| | |
|---|---|
| AGAA AGT ACA TAT GTC | (SEQ ID NO:1) |

In this example, the 16-mer contains 14 different 3-mer sequences (AGA GAA AAA AAG AGT GTA TAC ACA CAT ATA TAT ATG TGT GTC), since none of the 3-mers is identical. To prevent binding of code components at the wrong location, at least 18 different types (=14+4) of uniquely tagged 3-mer code sequences are needed in order to distinguishably tag all possible 4-mer probe sequences 550. (The number of unique code components required may be calculated as equal to ((2 times n)+(n times m)−m+1).) With the specific container sequence 540 disclosed in SEQ ID NO:1, only 4 tagged 3-mers are required—TCA, TGT, ATG and CAG. Each tagged 3-mer can bind at one and only one site on the template 500. Because the tagged oligonucleotides 520 are complementary in sequence to the container section 540, an “A” in the container section 540 binds to a “T” in the oligonucleotide 520, while a “G” will bind to a “C” and vice-versa. Any changes in the sequence of the probe section 550 will require corresponding changes to be made in the container section 540 sequence. For example, if the probe sequence 550 is changed from AGAA to AGTA, the container sequence 540 must be changed also, since the AGT in the probe 550 overlaps with the AGT in the container 540. A possible new template 500 sequence is shown in SEQ ID NO:2 below.

| | |
|---|---|
| AGTA AGA ACA TAT GTC | (SEQ D NO:2) |

The corresponding oligonucleotide 520 sequences would be TCT TGT ATA and CAG. Again, each binds at only one site in the container section 540 and cannot bind to the probe section 550. To allow for unique tagging of all possible 4-mer probe sequences 540 requires 18 different 3-mer tagged oligonucleotides 520, which is far less than the 64 tagged 3-mers 520 which would be required to generate all possible 3-mer sequences using known methods, such as sequencing by hybridization using complete probe libraries. The use of only 18 out of 64 possible 3-mers also avoids problems with using oligonucleotide 520 sequences that can potentially hybridize to each other.

The tagged oligonucleotides 520 (or code components) may be prepared in advance before barcode 530 synthesis and may be purified and stored. A given set of m-mers may be used to prepare barcodes 530 for any needed probe 550 sequence. This greatly improves the efficiency of probe 550 preparation, compared to existing methods wherein each tagged probe 550 molecule is separately prepared and individually labeled and purified. The modular system disclosed herein exhibits great efficiency of labeling compared to known methods.

Normally, attaching a signal (label) component to a nucleic acid strand involves the use of labeled nucleotides or a post-synthesis labeling process, both of which may cause problems. DNA polymerases typically cannot efficiently process labeled nucleotides for incorporation into oligonucleotides 520 or nucleic acids. When multiple signal components are to be added to a single nucleic acid strand, the efficiency of incorporation decreases dramatically. DNA strands with more than 1 or 2 labels require a large amount of starting material and substantial purification of the labeled molecule to separate it from unlabeled or partially labeled molecules, due to the low incorporation efficiency. The use of multiple short tagged oligonucleotides 520 disclosed herein avoids such problems.

When barcode 530 molecules are designed for specific target molecules, the structure and signal component of the barcode 530 is fixed and the barcode 530 is only suited for one purpose. If barcodes 530 are needed for other targets, each must be prepared from the start. The present modular system, using short tagged oligonucleotides 520 which may be prepared in advance and stored, greatly improves the flexibility, simplicity and speed of barcode 530 production for any target. The reduced number of uniquely tagged code components required also decreases cost and improves the efficiency of detection, since it reduces the number of distinguishable tagged probes 550 that must be prepared and identified.

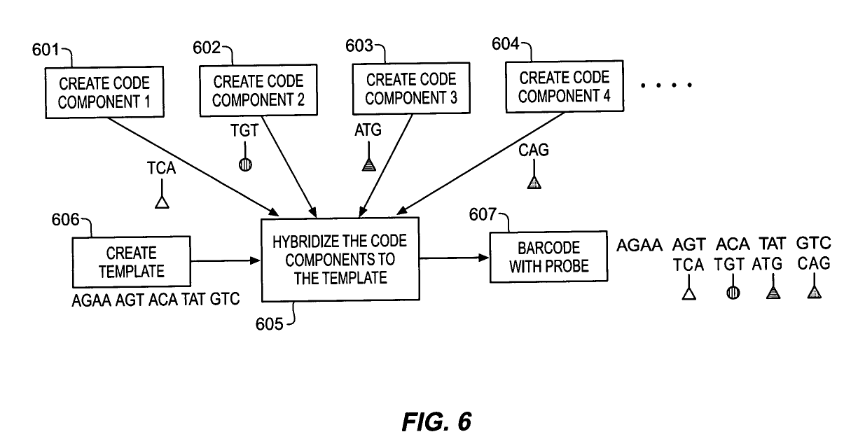

FIG. 6 illustrates an exemplary method for generating a barcode, such as the barcodes discussed above. For example, code components 601 602 603 604 may be generated by synthesizing short oligonucleotides (e.g. 3-mer) and linking a tag to the oligonucleotide or incorporating a nucleotide already modified by a tag. The tags linked to the oligonucleotide are not limited to Raman tags. For example, fluorescent, nanoparticle, nanotube, fullerenes and quantum dot tags may also be attached to the oligonucleotide. The mode of attachment to the oligonucleotide may vary. The tag may be directly attached to the oligonucleotide or may be attached through a branch structure. Various methods for producing tagged oligonucleotides of use as code components 601 602 603 604 are well known in the art. A template 606 having an extended probe region may be created that is complementary in sequence to the tagged code components 601 602 603 604. The tagged components 601 602 603 604 are hybridized 605 to the template 606 either individually or as a mixture. The resulting barcode 607 includes a double-stranded region with detectable tags and a single-stranded probe region for binding to target molecules.

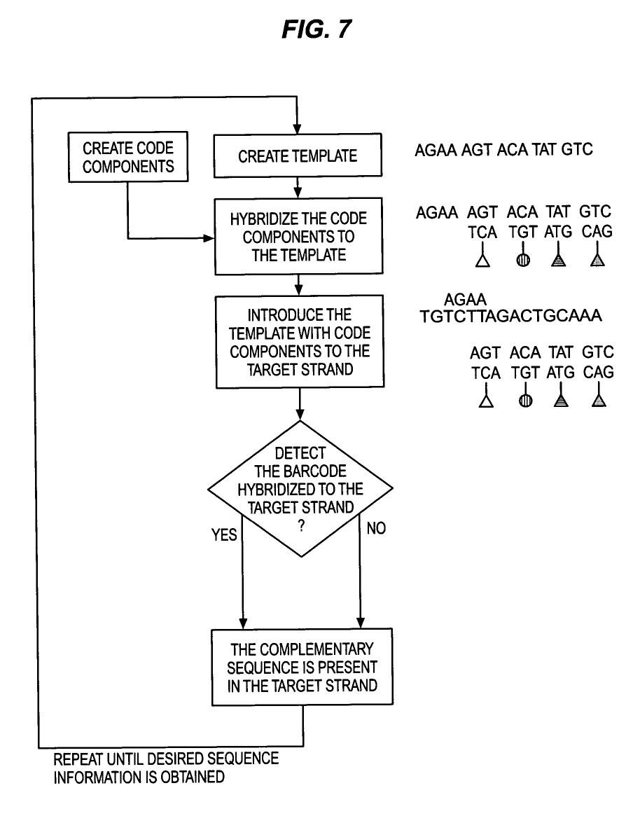

FIG. 7 illustrates a schematic for generation and use of barcodes. Barcodes may be generated by creating a template molecule and code components as discussed above. The code components may be hybridized to the template as discussed above, producing a barcode. Once a barcode is generated, it may be used for a variety of purposes, such as to detect an oligonucleotide, nucleic acid or other target molecule in a sample or for sequencing a nucleic acid molecule. As shown in FIG. 7, nucleic acid targets may be sequenced by repetitive exposure of the target molecule to solutions comprising one or more barcodes. Hybridization of the barcode to the target indicates the present of a complementary sequence in the target strand. The process may be repeated, with exposure to different barcodes indicating the presence of different complementary sequences. As with the process of “shotgun” sequencing, some of the complementary sequences may overlap. The overlapping complementary sequences may be assembled into a complete target nucleic acid sequence.

The barcode may be introduced to a sample and binding to the target molecule detected by any known imaging modality, for example fluorescent microscopy, FTIR (Fourier transform infra-red) spectroscopy, Raman spectroscopy, surface plasmon resonance, and/or electron microscopy.

Polymeric Raman Label Barcodes by Covalent Bonding

In certain embodiments of the invention, polymeric Raman label barcodes may be generated. Generally, the polymeric Raman label will comprise a backbone moiety to which Raman tags are attached, directly or via spacer molecules. The backbone moiety may be comprised of any type of monomer suitable for polymerization, including but not limited to nucleotides, amino acids, monosaccharides or any of a variety of known plastic monomers, such as vinyl, styrene, carbonate, acetate, ethylene, acrylamide, etc. The polymeric Raman label may be attached to a probe moiety, such as an oligonucleotide, antibody, lectin or aptamer probe. Where the polymeric backbone is comprised of nucleotide monomers, attachment to an antibody probe would minimize the possibility of binding of both probe and backbone components to different target molecules. Alternatively, in certain embodiments of the invention using nucleotide monomers for the backbone, the sequence of nucleotides incorporated into the polymeric Raman label could be designed to be complementary to a target nucleic acid, allowing the probe function to be incorporated into the polymeric Raman label. Because a nucleotide-based backbone would itself produce a Raman emission spectrum that could potentially interfere with detection of attached Raman tags, in some embodiments a backbone component that produces little or no Raman emission signal may be used to optimize signal detection and minimize signal-to-noise ratio. The following section relates to polymeric Raman labels in general, without limitation as to the specific type of monomeric unit to be used.

Polymeric Raman label barcodes may be used for target molecule detection, identification and/or sequencing as discussed above. Current methods for probe labeling and detection exhibit various disadvantages. For example, probes attached to organic fluorescent tags offer high detection sensitivity but have low multiplex detection capability. Fluorescent tags exhibit broad emission peaks, and fluorescent resonant energy transfer (FRET) limits the number of different fluorescent tags that can be attached to a single probe molecule, while self-quenching reduces the quantum yield of the fluorescent signal. Fluorescent tags require multiple excitation sources if a probe contains more than one type of chromophore. They are also unstable due to photo-bleaching. Another type of potential probe tag is the quantum dot. Quantum dot tags are relatively large structures with multiple layers. In addition to being complicated to produce, the coating on quantum dots interferes with fluorescent emission. There are limits on the number of distinguishable signals that can be generated using quantum dot tags. A third type of probe label consists of dye-impregnated beads. These tend to be very large in size, often larger than the size range of the probe molecule. Detection of dye-impregnated beads is qualitative, not quantitative.

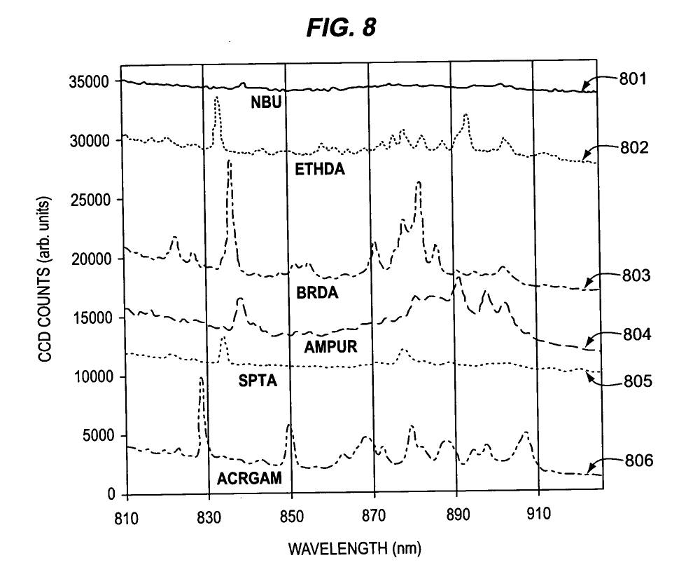

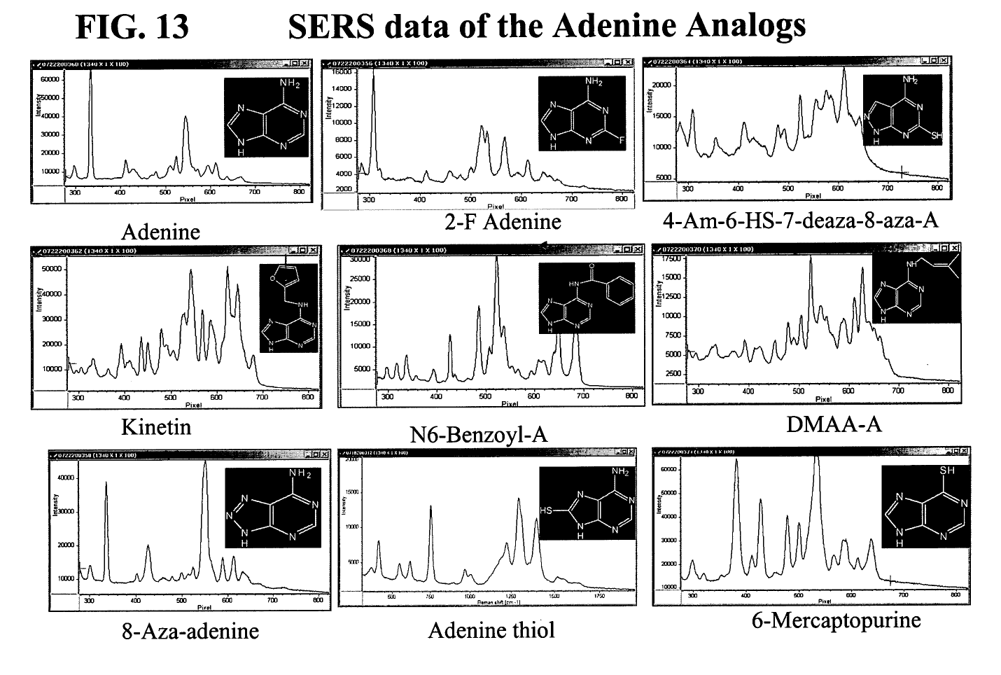

Raman labels offer the advantage of producing sharp spectral peaks, allowing a greater number of distinguishable labels to be attached to probes. The use of surface enhanced Raman spectroscopy (SERS) or similar techniques allows a sensitivity of detection comparable to fluorescent tags. The emission spectra of exemplary Raman tag molecules are shown in FIG. 8. As can be seen from the figure, the Raman tag molecules provide a multiplicity of distinguishable spectra. FIG. 8 represents the spectra of the following Raman tag molecules: NBU (oligonucleotide 5′-(T)20-deoxyNebularine-T-3′); ETHDA (oligonucleotide 5′-(T)20-(N-ethyldeoxyadenosine)-T-3′); BRDA (oligonucleotide 5′-(T)20-(8-Bromoadenosine)-T-3′); AMPUR (oligonucleotide 5′-(T)20-(2-Aminopurine)-T-3′); SPTA (oligonucleotide 5′-ThiSS-(T) 20-A-3′); and ACRGAM (oligonucleotide 5′-acrydite-(G)20-Amino-C₇₋₃′). FIG. 13 represents SERS spectra of some of the nucleic acid analogs of one nucleic acid, adenine, compared to the nuclei acid spectra itself: Adenine; 2-F Adenine, 4-Am-6-HS-7-deaza-8-aza-Adenine; kinetin; N6-Benzoyl-Adenine; DMAA-A; 8-Aza-Adenine; Adenine thiol and a purine derivative, 6-Mercaptopurine. Table 1 lists other tag molecules of potential use in Raman spectroscopy. The skilled artisan will realize that the Raman tags of use are not limited to those disclosed herein, but may include any known Raman tag that may be attached to a probe and detected. Many such Raman tags are known in the art (see, e.g., www.glenres.com).

TABLE 1
Examples of Raman Tag Molecules

2′,3′-ddA-5′-CE Phosphoramidite
2′-deoxyAdenosine a-thiotriphosphate (15 mM) (2′dATTPaS)
2′-Fluoro-Adenosine a-thiotriphosphate (10 mM) (2′-F-ATTPaS)
2′-OMe-A-CE Phosphoramidite
2′-OMe-A-Me Phosphonamidite
2′-OMe-A-RNA
2′-OMe-Adenosine a-thiotriphosphate (20 mM) (2′-O-Me-ATTPaS)
2′-OMe-Pac-A-CE Phosphoramidite
2-Amino-dA-CE Phosphoramidite
2-Aminopurine riboside a-thiotriphosphate (20 mM) (2-AP-TTPaS)
2-F-dA-CE Phosphoramidite
3′-A-TOM-CE Phosphoramidite
3′-dA-CE Phosphoramidite
3′-dA-CPG
7-Deaza-Adenosine a-thiotriphosphate (1 mM) (7-DATTPaS)
7-deaza-dA CE Phosphoramidite
8-Amino-dA-CE Phosphoramidite
8-Br-dA-CE Phosphoramidite
8-oxo-dA-CE Phosphoramidite
A-TOM-CE Phosphoramidite
A-RNA-TOM-CPG
Adenosine a-thiotriphosphate (0.5 mM) (ATTPaS)
Bz-A-CE Phosphoramidite
Bz-A-RNA-CPG
dA-5′-CE Phosphoramidite
dA-5′-CPG
dA-CE Phosphoramidite
dA-CPG 1000
dA-CPG 2000
dA-CPG 500
dA-High Load-CPG
dA-Me Phosphonamidite
dA-Q-CPG 500
Diaminopurine riboside a-thiotriphosphate (0.25 mM) (DTTPaS)

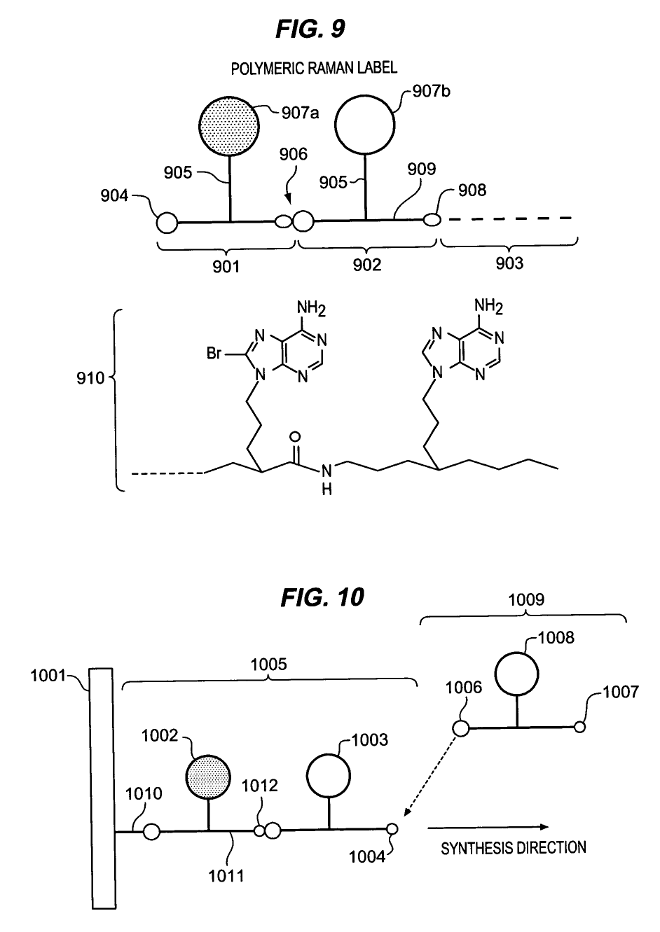

FIG. 9 illustrates an exemplary method for generating barcodes by linking together two or more Raman tagged monomeric units 901, 902 to form a polymeric Raman label. The polymeric Raman label may be attached to a probe moiety for binding to and detection of a target molecule. A polymeric Raman label may comprise a first monomeric unit 901 attached by a covalent bond 906 to a second monomeric unit 902. Where greater signal complexity is needed, additional monomeric units 903 may be attached. The monomeric units 901 902 may include one or more Raman tag moieties 907a, 907b, directly attached or attached by a spacer 905 to the backbone 909. The spacer 905 may comprise, for example, five or more carbon atoms. The length of a spacer 905 may vary, for example, between 2 to 30, 2 to 20 or 3 to 15 carbon atoms long. The most effective spacer 905 would be flexible, such as an aliphatic carbon (e.g. through aminocaproic acid), a peptide chain (e.g. linked through a side chain of lysine) or polyethylene glycol (e.g., phosphoramidite). The spacer 905 may contain carbon, nitrogen, sulfur and/or oxygen atoms. Various methods for producing and cross-linking tagged monomeric units 901 902 are known in the art. Various tagged monomeric units may also be obtained from commercial sources (e.g., Molecular Probes, Eugene, Oreg.).

As illustrated in FIG. 9, a barcode may be formed by covalently linking one monomeric unit 901 to another monomeric unit 902 through functional groups 904, 908. The functional groups 904, 908 may include for example biotin, amino groups, aldehyde groups, thiol groups or any other reactive group known in the art. Each monomeric unit 901, 902 has at least two functional groups 904, 908, one attached to each end of the monomer. Prior to cross-linking, one functional group 904, 908 may be activated (deprotected) to attach to another monomeric unit 901, 902, while a second functional group 904, 908 remains protected from interaction or blocked (e.g. by a chemical modification). Each end of a monomeric unit 901, 902 is capable of binding to another monomeric unit 901, 902 when activated. In various embodiments, a polymeric Raman label may comprise between 2 to 30, 4 to 20 or 5 to 15 monomeric units 901, 902 (e.g. nucleotides, amino acids, plastic monomers, etc). An example of a polymeric Raman label 910 comprised of two monomeric units 901, 902 linked together by a covalent bond 906 is illustrated. The Raman tags 907a 907b are shown attached via a spacer molecule 905 to the backbone 909. The monomeric units 901, 902 are attached to each other by a covalent bond 906, in this instance by an amide linkage formed, for example, by carbodiimide catalyzed reaction of a carboxyl group with a primary amino group.

It is contemplated that the Raman tag 907a 907b may comprise one or more double bonds, for example carbon to nitrogen double bonds. It is also contemplated that the Raman tags 907a 907b may comprise a ring structure with side groups attached to the ring structure. The side groups may include but are not limited to nitrogen atoms, oxygen atoms, sulfur atoms, and halogen atoms as well as carbon atoms and hydrogen atoms. Side groups that increase Raman signal intensity for detection are of particular use. Effective side groups include compounds with conjugated ring structures, such as purines, acridines, Rhodamine dyes and Cyanine dyes. The overall polarity of a polymeric Raman label is contemplated to be hydrophilic, but hydrophobic side groups may be included.

An exemplary method to generate polymeric Raman labels is shown in FIG. 10. A solid support 1001 may be used to anchor the growing polymeric Raman label. The support 1001 can comprise, for example, porous glass beads, plastics (including but not limited to acrylics, polystyrene, copolymers of styrene and other materials, polypropylene, polyethylene, polybutylene, polyurethanes, TeflonJ, etc.), polysaccharides, nylon, nitrocellulose, composite materials, ceramics, plastic resins, silica, silica-based materials, silicon, modified silicon, carbon, metals, inorganic glasses, optical fiber bundles or any other type of known solid support. One or more linker molecules 1010 (such as a carbon atom chain) may be attached to the support 1001. The length of the linker molecule 1010 may vary. For example, the linker 1010 may be 2-50 atoms in length. Various types of linkers 1010 of use are discussed above. It is contemplated that more than one length or type of linker molecule 1010 may be attached to the solid support 1001. The linker 1010 serves as an attachment site to grow a polymeric Raman label by stepwise attachment of monomeric units 1009. FIG. 10 shows an attached component 1005 of a polymeric Raman label comprising two monomers.

Each monomeric unit 1009 to be attached comprises two functional groups 1006, 1007, as discussed above, one on each end of the monomeric unit 1009. Addition of monomeric units 1009 occurs by the selective activation of the functional group 1006 on the leading end of the monomeric unit 1009. The activated functional group 1006 may be attached to another activated functional group 1004 at the growing end of the component 1005. Methods for chemical synthesis of polymers are known in the art and may include, for example, phosphoramidite synthesis of oligonucleotides and/or solid-phase synthesis of peptides. Methods of protecting and deprotecting functional groups 1004, 1006, 1007 are also well known in the art, as in the techniques of oligonucleotide or peptide synthesis.

Each successive monomeric unit 1009 may be introduced in solution, for example suspended in acetonitrile or other solvent. A functional group 1006 on the leading end of a first monomeric unit 1009 can bind to a linker molecule 1010. Once the first monomeric unit 1009 is attached to a linker molecule 1010, a functional group 1007 attached to the other end of the monomeric unit 1009 may be deprotected by chemical treatment (e.g. ammonium hydroxide) in order for another monomeric unit 1009 to bind. The second monomeric unit 1009 to be added may comprise an activated functional group 1006 and a protected functional group 1007, allowing for directional attachment of the monomeric unit 1009. After incorporation of the monomeric unit 1009 into the growing component 1005 of the polymeric Raman label, the protected functional group 1004 may be deprotected and another monomeric unit 1009 added. Additional rounds of this process may continue until a polymeric Raman label of appropriate length is generated.

It is contemplated that several different monomeric units 1009 may be added to the solid support 1001 at any given time to generate different polymeric Raman labels. In the latter case, the different polymeric Raman labels may be separated after synthesis if appropriate. The length of the polymeric Raman label will vary depending upon the number of monomeric units 1009 incorporated, but each polymeric label will contain two or more monomeric units 1009.

In various embodiments of the invention, a polymeric Raman label may contain 1, 2, 3, 4, 5, 6, 7, 8, 9, 10, 11, 12, 13, 14, 15, 16, 17, 18, 19, 20, 21, 22, 23, 24, 25 or more Raman tags 1002, 1003, 1008. The individual Raman tags 1002, 1003, 1008 attached to a single polymeric Raman label may each be different. Alternatively, a polymeric Raman label may contain two or more copies of the same Raman tag 1002, 1003, 1008. To maximize the number of distinguishable polymeric Raman labels, it is contemplated that where multiple Raman tags 1002, 1003, 1008 are incorporated into a single polymeric Raman label they will generally be different. As discussed above, Raman tags 1002, 1003, 1008 may be attached directly to the backbone 1011 of the polymeric Raman label 1009 or may be attached via a spacer molecule.

Polymeric Raman labels provide greater variety for spectral differentiation than monomeric labels, while allowing for the sensitivity of Raman spectroscopic detection. The use of multiple Raman tags 1002, 1003, 1008 attached to a single polymeric Raman label allows for a very large number of distinguishable polymeric Raman labels to be produced. A 4-mer polymeric Raman label made from 10 different possible tagged monomeric units 1009 would generate over 5000 distinguishable Raman signatures. With 15 different tagged monomeric units 1009, over 30,000 distinguishable Raman signatures would result. Over 50,000 distinguishable Raman signatures may be generated with only 10 to 20 different tagged monomeric units 1009. Since the size of a monomeric unit 1009 is about the same as a nucleotide (approximately 1000 daltons), the average size of a 4-mer Raman label would be about 4000 Daltons. Therefore, polymeric Raman labels would allow probe-target binding with little steric hindrance.

In some embodiments of the invention, the monomeric units 1009 incorporated into the polymeric Raman label may have a spacer branch attached to the backbone, with another reactive group 1004, 1006, 1007 attached to the spacer branch. The reactive group 1004, 1006, 1007 may be protected or blocked during synthesis of the polymer. Raman tags 1002, 1003, 1008 may be attached to the deprotected spacer branch after polymer synthesis, or after incorporation of the monomeric unit 1009 into the growing polymer 1005.

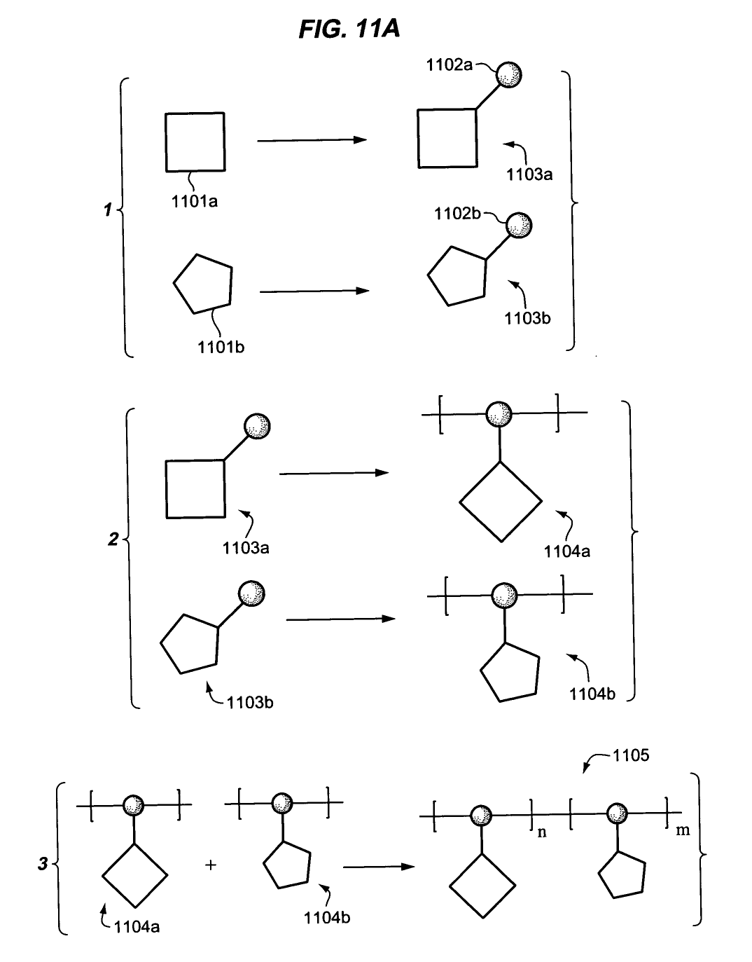

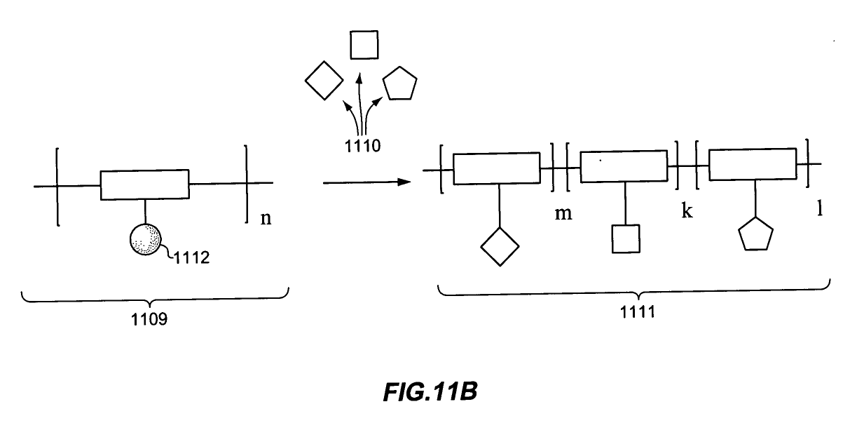

In certain embodiments of the invention, illustrated in FIG. 11A, polymeric Raman labels 1105 may be generated without a support. A Raman tag 1101a 1101b may be chemically altered to add a functional group 1102a 1102b, for example biotin, amino groups, aldehyde groups, thiol groups or any other type of reactive group, to generate a functionalized Raman tag (monomeric unit) 1103a 1103b. The monomeric units 1103a 1003b may then be subjected to polymerization to generate subpolymeric units 1104a 1104b, each comprising a predetermined number of monomeric units. The subpolymeric units 1004a 1004b may be mixed together in a predetermined ratio (e.g 1:1; 1:2, 1:10 etc.) and subjected to additional polymerization to produce the final polymeric Raman label 1105. In the example shown, the polymeric Raman label 1105 comprises “n” copies of one type of monomeric unit 1103a and “m” copies of a second type of monomeric unit 1103b.

FIG. 11B illustrates an alternative method for generating polymeric Raman labels 1111 without a support. In this case, one or more polymers 1109 may contain reactive side groups 1112 attached to spacers extending from the backbone. The reactive side groups 1112 may be attached to one or more different Raman tags 1110 to create a polymeric Raman label 1111. The reactive side groups 1112 may include polylysine treated to convert the amine side chains to maleimide residues (polymaleic anhydride), which can react with HS (hydrogen sulfate) functionalized Raman tags 1110. Alternatively, the side groups 1112 may comprise the amine groups of poly(allylamine), which may react with NHS ester functionalized Raman tags 1110. The side groups 1112 may also comprise the carboxylic acid groups of succinylated polylysine or synthetic oligonucleotides with amino or carboxylic acid groups. Carboxylate side groups 1112 may be attached to Raman tags 1110, for example using carbodiimide mediated cross-linking.

The polymer backbones may be formed from organic structures, for example any combination of nucleic acid, peptide, polysaccharide, and/or chemically derived polymers. The backbone of a polymeric Raman label 1111 may be formed by phosphodiester bonds, peptide bonds, and/or glycosidic bonds. For example, standard phosphoramidite chemistry may be used to make backbones comprising DNA chains. Other methods for making phosphodiester-linked backbones are known, such as polymerase chain reaction (PCR™) amplification. The ends of the backbone may have different functional groups, for example, biotins, amino groups, aldehyde groups or thiol groups. These functionalized groups may be used to link two or more subpolymeric units together. For example, a polymeric Raman label 1111 may comprise “m” copies of a first monomeric unit, “k” copies of a second monomeric unit, and “1” copies of a third monomeric unit. Once the polymer backbone is synthesized to the desired length, two or more different Raman tags 1110 may be introduced sequentially or simultaneously to bind to reactive side groups 1112, thereby generating the polymeric Raman label. The monomeric unit is not restricted to Raman tags 1110. Other tags, for example fluorescent, nanoparticle, nanotube, fullerenes or quantum dot tags may be attached to one or more monomeric units in order to diversify the polymeric Raman label 1111. Generally, the majority of the tags 1110 of the monomeric units will be Raman tags 1110. More than one polymeric Raman label 1111 may be joined to generate an even longer product.

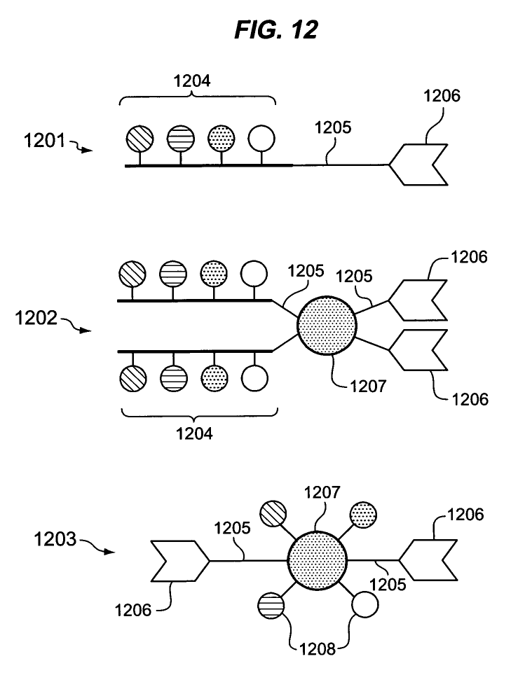

In certain embodiments of the invention, illustrated in FIG. 12, any of the polymeric Raman labels disclosed above may be linked to a probe 1206. Examples of probe molecules 1206 may include but are not limited to oligonucleotides, nucleic acids, antibodies, antibody fragments, binding proteins, receptor proteins, peptides, lectins, substrates, inhibitors, activators, ligands, hormones, cytokines, etc.

Various exemplary structures 1201 1202 of polymeric Raman labels 1204 may comprise covalently linked monomeric units, with a backbone and one or more Raman tags attached directly or via a spacer molecule to the backbone. The polymers 1204 may be attached to a probe 1206 through a linker 1205 or direct covalent bond 1205. Alternatively, the polymeric Raman labels 1204 may be attached to one or more probe moieties 1206 indirectly, via attachment to a nanoparticle 1207. Various methods for cross-linking molecules to nanoparticles are known in the art, and any such known method may be used. For example, by crosslinking a carboxyl group with an amine group in the presence of EDAC (1-Ethyl-3-(3-dimethylaminopropyl)carbodiimide). As shown in an exemplary structure 1202, more than one polymeric Raman label 1204 may be attached to a single nanoparticle 1207. The nanoparticle 1207 may then be attached to one or more probe molecules 1206. The advantage of this type of structure is that more than one target molecule may be identified using a single polymeric Raman label 1204. Alternatively, multiple copies of the same target molecule may be bound if the nanoparticle 1207 is attached to multiple copies of the same probe molecule 1206. Other advantages include a greater chance to capture target molecules since there are more probe molecules 1206 attached to the Raman label and a separation of free and Raman label-bound target molecules is made easier in solution detection applications since Raman label 1202 can be isolated by centrifugation, filtration, or electrophoresis.

In an alternative structure 1203, monomeric Raman tags 1208 may be attached to a nanoparticle 1207, either directly or via a spacer molecule 1205. One or more probe molecules may be attached to the same nanoparticle 1207 directly or by a spacer 1205. This allows for the formation of multiple Raman tags 1208 attached to a probe 1206, without the need for preliminary synthesis of a polymer 1204. The advantage of this structure 1203 is that the nanoparticle 1207 has a greater surface area, allowing more probe molecules 1206 and Raman tags 1208 to bind while providing decreased steric hindrance between molecules.

A large variety of polymeric Raman label barcodes may be created using relatively few monomeric units. The generation of polymeric Raman labels allows a greater flexibility and sensitivity in barcode generation while utilizing relatively few Raman tags.

Nucleic Acids

Nucleic acid molecules to be sequenced may be prepared by any standard technique. In one embodiment, the nucleic acids may be naturally occurring DNA or RNA molecules. Where RNA is used, it may be desired to convert the RNA to a complementary cDNA. Virtually any naturally occurring nucleic acid may be prepared and sequenced by the methods of the present invention including, without limit, chromosomal, mitochondrial or chloroplast DNA or messenger, heterogeneous nuclear, ribosomal or transfer RNA. Methods for preparing and isolating various forms of cellular nucleic acids are known (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). Non-naturally occurring nucleic acids may also be sequenced using the disclosed methods and compositions. For example, nucleic acids prepared by standard amplification techniques, such as polymerase chain reaction (PCR™) amplification, could be sequenced within the scope of the present invention. Methods of nucleic acid amplification are well known in the art.

Nucleic acids may be isolated from a wide variety of sources including, but not limited to, viruses, bacteria, eukaryotes, mammals, and humans, plasmids, M13, lambda phage, PI artificial chromosomes (PACs), bacterial artificial chromosomes (BACs), yeast artificial chromosomes (YACs) and other cloning vectors.

Methods of Nucleic Acid Immobilization

In various embodiments, nucleic acid molecules may be immobilized by attachment to a solid surface. Immobilization of nucleic acid molecules may be achieved by a variety of methods involving either non-covalent or covalent attachment to a support or surface. In an exemplary embodiment, immobilization may be achieved by coating a solid surface with streptavidin or avidin and binding of a biotinylated polynucleotide. Immobilization may also occur by coating a polystyrene, glass or other solid surface with poly-L-Lys or poly L-Lys, Phe, followed by covalent attachment of either amino- or sulfhydryl-modified nucleic acids using bifunctional crosslinking reagents. Amine residues may be introduced onto a surface through the use of aminosilane.

Immobilization may take place by direct covalent attachment of 5′-phosphorylated nucleic acids to chemically modified polystyrene surfaces. The covalent bond between the nucleic acid and the solid surface is formed by condensation with a water-soluble carbodiimide. This method facilitates a predominantly 5′-attachment of the nucleic acids via their 5′-phosphates.

DNA is commonly bound to glass by first silanizing the glass surface, then activating with carbodiimide or glutaraldehyde. Alternative procedures may use reagents such as 3-glycidoxypropyltrimethoxysilane or aminopropyltrimethoxysilane (APTS) with DNA linked via amino linkers incorporated either at the 3′ or 5′ end of the molecule during DNA synthesis. DNA may be bound directly to membranes using ultraviolet radiation. Other methods of immobilizing nucleic acids are known.

The type of surface to be used for immobilization of the nucleic acid is not limited. In various embodiments, the immobilization surface may be magnetic beads, non-magnetic beads, a planar surface, a pointed surface, or any other conformation of solid surface comprising almost any material, so long as the material will allow hybridization of nucleic acids to probe libraries.

Bifunctional cross-linking reagents may be of use in various embodiments. Exemplary cross-linking reagents include glutaraldehyde, bifunctional oxirane, ethylene glycol diglycidyl ether, and carbodiimides, such as 1-ethyl-3-(3-dimethylaminopropyl) carbodiimide.

In certain embodiments a capture oligonucleotide may be bound to a surface. The capture oligonucleotide will hybridize with a specific nucleic acid sequence of a nucleic acid template. A nucleic acid may be released from a surface by restriction enzyme digestion, endonuclease activity, elevated temperature, reduced salt concentration, or a combination of these and similar methods.

Protein Purification

In certain embodiments a protein or peptide may be isolated or purified. In one embodiment, these proteins may be used to generate antibodies for tagging with any of the illustrated barcodes (eg. polymeric Raman label). Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, HPLC (high performance liquid chromatography) FPLC (AP Biotech), polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. One of the more efficient methods of purifying peptides is fast performance liquid chromatography (AKTA FPLC) or even HPLC.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulfate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low-pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically binds. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

Peptide Mimetics

Another embodiment for the preparation of polypeptides according to the invention is the use of peptide mimetics for monoclonal antibody production. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

Fusion Proteins

Other embodiments of the invention concern fusion proteins. These molecules generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In certain embodiments, a fusion proteins comprises a targeting peptide linked to a therapeutic protein or peptide. It is contemplated that within the scope of the present invention virtually any protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

Synthetic Peptides

Because of their relatively small size, the peptides identified after a fungal selection process may be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

Exemplary Applications

Nucleic Acid Sequencing

In particular embodiments, barcodes formed as disclosed herein may be used to sequence target nucleic acid molecules. Methods for sequencing by hybridization are known in the art. One or more tagged barcodes comprising probes of known sequence may be allowed to hybridize to a target nucleic acid sequence. Binding of the tagged barcode to the target indicates the presence of a complementary sequence in the target strand. Multiple labeled barcodes may be allowed to hybridize simultaneously to the target molecule and detected simultaneously. In alternative embodiments, bound probes may be identified attached to individual target molecules, or alternatively multiple copies of a specific target molecule may be allowed to bind simultaneously to overlapping sets of probe sequences. Individual molecules may be scanned, for example, using known molecular combing techniques coupled to a detection mode. (See, e.g., Bensimon et al., Phys. Rev. Lett. 74:4754-57, 1995; Michalet et al., Science 277:1518-23, 1997; U.S. Pat. Nos. 5,840,862; 6,054,327; 6,225,055; 6,248,537; 6,265,153; 6,303,296 and 6,344,319.)

It is unlikely that a given target nucleic acid will hybridize to contiguous probe sequences that completely cover the target sequence. Rather, multiple copies of a target may be hybridized to pools of tagged oligonucleotides and partial sequence data collected from each. The partial sequences may be compiled into a complete target nucleic acid sequence using publicly available shotgun sequence compilation programs. Partial sequences may also be compiled from populations of a target molecule that are allowed to bind simultaneously to a library of barcode probes, for example in a solution phase.

Target Molecule Detection, Identification and/or Quantification

In certain embodiments, target molecules in a sample may be detected, identified and/or quantified by binding to barcodes. Tagged barcodes designed to bind to specific targets may be prepared as discussed above. The targets are not limited to nucleic acids, but may also include proteins, peptides, lipids, carbohydrates, glycolipids, glycoproteins or any other potential target for which a specific probe may be prepared. As discussed above, antibody or aptamer probes may be incorporated into barcodes and used to identify any target for which an aptamer or antibody can be prepared. The presence of multiple targets in a sample may be assayed simultaneously, since each barcode may be distinguishably labeled and detected. Quantification of the target may be performed by standard techniques, well known in spectroscopic analysis. For example, the amount of target bound to a tagged barcode may be determined by measuring the signal intensity of bound barcode and comparison to a calibration curve prepared from known amounts of barcode standards. Such quantification methods are well within the routine skill in the art.

Array Chemistry

Beads (e.g. microspheres), carrying different chemical functionalities (e.g., different binding specificities) may be mixed together. The ability to identify the functionality of each bead may be achieved using an optically interrogatable encoding scheme (an “optical signature”). For example, an optical signature may be generated using polymeric Raman labels as discussed above. A substrate, such as a chip or a microtiter plate, may comprise a patterned surface containing individual sites that can bind to individual beads. This allows the synthesis of the probes (i.e. nucleic acids, aptamers or antibodies) to be separated from their placement on the array. The probes may be synthesized, attached to the beads and the beads randomly distributed on a patterned surface. Since the beads are first coded with an optical signature, the resulting array can later be “decoded”. That is, a correlation between the location of an individual site on the array with the bead or probe located at that particular site can be made. Because the beads may be randomly distributed on the array, this results in a fast and inexpensive process compared to either in situ synthesis or spotting techniques for array production.

Array compositions may include at least a first substrate with a surface comprising individual sites. The size of the array will depend on the end use of the array. Arrays containing from about 2 different agents (i.e. different beads) to many millions of different agents can be made. Generally, the array will comprise from two different beads to as many as a billion or more, depending on the size of the beads and the substrate. Thus, very high density, high density, moderate density, low density or very low density arrays may be made. Some ranges for very high-density arrays are from about 10,000,000 to about 2,000,000,000 sites per array. High-density arrays range from about 100,000 to about 10,000,000 sites. Moderate density arrays range from about 10,000 to about 50,000 sites. Low-density arrays are generally less than 10,000 sites. Very low-density arrays are less than 1,000 sites.

In some embodiments of the invention, multiple substrates may be used, either of different or identical compositions. Thus for example, large arrays may include a plurality of smaller substrates. By “substrate” or “solid support” is meant any material that can be modified to contain discrete individual sites appropriate for the attachment or association of beads and amenable to at least one detection method. In general, the substrates allow optical detection and do not appreciably interfere with signal emissions.

The sites comprise a pattern, i.e. a regular design or configuration, or may be randomly distributed. A regular pattern of sites may be used such that the sites may be addressed in an X-Y coordinate plane. The surface of the substrate may be modified to allow attachment of microspheres at individual sites. Thus, the surface of the substrate may be modified such that discrete sites are formed that can only have a single associated bead. In one embodiment, the surface of the substrate may be modified to contain wells, i.e. depressions in the surface of the substrate. This may be done using a variety of known techniques, including, but not limited to, photolithography, stamping techniques, molding techniques and microetching techniques. As will be appreciated by those in the art, the technique used will depend on the composition and shape of the substrate. Alternatively, the surface of the substrate may be modified to contain chemically derived sites that can be used to attach microspheres and/or beads to discrete locations on the substrate. The addition of a pattern of chemical functional groups, such as amino groups, carboxy groups, oxo groups and thiol groups may be used to covalently attach microspheres, which generally contain corresponding reactive functional groups or linker molecules.

Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon® may all be used. The bead size may range from nanometers, i.e. 100 nm, to millimeters, i.e. 1 mm, with beads from about 0.2 micron to about 200 microns, and from about 0.5 to about 5 micron, although in some embodiments smaller beads may be used.

The compositions may be used to detect the presence of a particular target analyte, for example, a nucleic acid, oligonucleotide, protein, enzyme, antibody or antigen. The compositions may also be used to screen bioactive agents, i.e. drug candidates, for binding to a particular target or to detect agents like pollutants. As discussed above, any analyte for which a probe moiety, such as a peptide, protein, oligonucleotide or aptamer, may be designed can be used in combination with the disclosed barcodes.

Bioactive agents can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification and/or amidification to produce structural analogs.

Bioactive agents may comprise naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening the systems described herein. For example libraries of bacterial, fungal, viral, and mammalian proteins may be generated for screening purposes

The bioactive agents may be peptides of from about 5 to about 30 amino acids or about 5 to about 15 amino acids. The peptides may be digests of naturally occurring proteins or random peptides. Since generally random peptides (or random nucleic acids) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized bioactive agents.

Alternatively, the bioactive agents may be nucleic acids. The nucleic acids may be single stranded or double stranded, or a mixture thereof. The nucleic acid may be DNA, genomic DNA, cDNA, RNA or a hybrid, where the nucleic acid contains any combination of deoxyribo- and ribonucleotides, and any combination of bases, including uracil, adenine, thymine, cytosine, guanine, inosine, xanthanine, hypoxanthanine, isocytosine, isoguanine, and basepair analogs such as nitropyrrole and nitroindole, etc.

The applications of the barcodes disclosed herein are not limited to the preceding uses, but may include any use for which target detection, identification and/or quantification may be involved. Non-limiting applications including detection of single-nucleotide polymorphisms (SNPs), detection of genetic mutations, disease diagnosis, forensic analysis, detection of environmental contaminants and/or pathogens, clinical diagnostic testing and a wide variety of other applications known in the art.

Probe Preparation

Oligonucleotide Probes

Methods for oligonucleotide synthesis are well known in the art and any such known method may be used. For example, oligonucleotides may be prepared using commercially available oligonuclotide synthesizers (e.g., Applied Biosystems, Foster City, Calif.). Nucleotide precursors attached to a variety of tags may be commercially obtained (e.g. Molecular Probes, Eugene, Oreg.) and incorporated into oligonucleotides. Alternatively, nucleotide precursors may be purchased containing various reactive groups, such as biotin, diogoxigenin, sulfhydryl, amino or carboxyl groups. After oligonucleotide synthesis, tags may be attached using standard chemistries. Oligonucleotides of any desired sequence, with or without reactive groups for tag attachment, may also be purchased from a wide variety of sources (e.g., Midland Certified Reagents, Midland, Tex.). Oligonucleotide probes may also be prepared by standard enzymatic process, for example using polymerase chain reaction (PCR™) amplification (e.g., Sambrook et al., Molecular Cloning: A Laboratory Manual, 2nd Ed., Cold Spring Harbor Press, Cold Spring Harbor, N.Y., 1989.; U.S. Pat. Nos. 5,279,721; 4,683,195; 4,683,202; 4,800,159; 4,883,750).

Aptamer Probes

Aptamers are oligonucleotides derived by an in vitro evolutionary process called SELEX (e.g., Brody and Gold, Molecular Biotechnology 74:5-13, 2000). The SELEX process involves repetitive cycles of exposing potential aptamers (nucleic acid ligands) to a target, allowing binding to occur, separating bound from free nucleic acid ligands, amplifying the bound ligands and repeating the binding process. After a number of cycles, aptamers exhibiting high affinity and specificity against virtually any type of biological target may be prepared. Because of their small size, relative stability and ease of preparation, aptamers may be well suited for use as probes. Since aptamers are comprised of oligonucleotides, they can easily be incorporated into nucleic acid type barcodes. Methods for production of aptamers are well known (e.g., U.S. Pat. Nos. 5,270,163; 5,567,588; 5,670,637; 5,696,249; 5,843,653). Alternatively, a variety of aptamers against specific targets may be obtained from commercial sources (e.g, Somalogic, Boulder, Colo.). Aptamers are relatively small molecules on the order of 7 to 50 kDa.

Antibody Probes

Methods of production of antibodies are also well known in the art (e.g., Harlow and Lane, Antibodies: A Laboratory Manual, Cold Spring Harbor Laboratory, Cold Spring Harbor, N.Y., 1988.) Monoclonal antibodies suitable for use as probes may also be obtained from a number of commercial sources. Such commercial antibodies are available against a wide variety of targets. Antibody probes may be conjugated to barcodes using standard chemistries, as discussed below.

The disclosed methods and compositions are not limiting as to the type of probe used, and any type of probe moiety known in the art may be attached to barcodes and used in the disclosed methods. Such probes may include, but are not limited to, antibody fragments, affibodies, chimeric antibodies, single-chain antibodies, ligands, binding proteins, receptors, inhibitors, substrates, etc.

Tags

In various embodiments of the invention, barcodes may be attached to one or more tag moieties to facilitate detection and/or identification. Any detectable tag known in the art may be used. Detectable tags may include, but are not limited to, any composition detectable by electrical, optical, spectrophotometric, photochemical, biochemical, immunochemical, or chemical techniques. Tags may include, but are not limited to, conducting, luminescent, fluorescent, chemiluminescent, bioluminescent and phosphorescent moieties, quantum dots, nanoparticles, metal nanoparticles, gold nanoparticles, silver nanoparticles, chromogens, antibodies, antibody fragments, genetically engineered antibodies, enzymes, substrates, cofactors, inhibitors, binding proteins, magnetic particles and spin label compounds. (U.S. Pat. Nos. 3,817,837; 3,850,752; 3,939,350; 3,996,345; 4,277,437; 4,275,149; and 4,366,241.)

Raman Tags

Non-limiting examples of Raman tags of use include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine and aminoacridine. These and other Raman tags may be obtained from commercial sources (e.g., Molecular Probes, Eugene, Oreg.).

Polycyclic aromatic compounds in general may function as Raman tags. Other tags that may be of use include cyanide, thiol, chlorine, bromine, methyl, phosphorus and sulfur. In certain embodiments, carbon nanotubes may be of use as Raman tags. The use of tags in Raman spectroscopy is known (e.g., U.S. Pat. Nos. 5,306,403 and 6,174,677).

Raman tags may be attached directly to barcodes or may be attached via various linker compounds. Nucleotides that are covalently attached to Raman tags are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman tags that contain reactive groups designed to covalently react with other molecules, for example nucleotides or amino acids, are commercially available (e.g., Molecular Probes, Eugene, Oreg.)

Fluorescent Tags

Fluorescent tags of potential use include, but are not limited to, fluorescein, 5-carboxyfluorescein (FAM), 2‘7’-dimethoxy-4‘5’-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-1-sulfonic acid (EDANS). Other potential fluorescent tags are known in the art (e.g., U.S. Pat. No. 5,866,336). A wide variety of fluorescent tags may be obtained from commercial sources, such as Molecular Probes (Eugene, Oreg.). Methods of fluorescent detection of tagged molecules are also well known in the art and any such known method may be used.

Luminescent tags of use include, but are not limited to, rare earth metal cryptates, europium trisbipyridine diamine, a europium cryptate or chelate, Th tribipyridine, diamine, dicyanins, La Jolla blue dye, allopycocyanin, allococyanin B, phycocyanin C, phycocyanin R, thiamine, phycoerythrocyanin, phycoerythrin R, an up-converting or down-converting phosphor, luciferin, or acridinium esters.

Nanoparticle Tags

Nanoparticles may be used as tags, for example where barcodes are to be detected by various modalities. Methods of preparing nanoparticles are known (e.g., U.S. Pat. Nos. 6,054,495; 6,127,120; 6,149,868; Lee and Meisel, J. Phys. Chem. 86:3391-3395, 1982). Nanoparticles may also be commercially obtained (e.g., Nanoprobes Inc., Yaphank, N.Y.; Polysciences, Inc., Warrington, Pa.). Although gold or silver nanoparticles are most commonly used as tags, any type or composition of nanoparticle may be attached to a barcode and used as a tag.

The nanoparticles to be used may be random aggregates of nanoparticles (colloidal nanoparticles). Alternatively, nanoparticles may be cross-linked to produce particular aggregates of nanoparticles, such as dimers, trimers, tetramers or other aggregates. Aggregates containing a selected number of nanoparticles (dimers, trimers, etc.) may be enriched or purified by known techniques, such as ultracentrifugation in sucrose solutions.

Modified nanoparticles suitable for attachment to barcodes are commercially available, such as the Nanogold® nanoparticles from Nanoprobes, Inc. (Yaphank, N.Y.). Nanogold® nanoparticles may be obtained with either single or multiple maleimide, amine or other groups attached per nanoparticle. Such modified nanoparticles may be attached to barcodes using a variety of known linker compounds.

Metallic Tags

Tags may comprise submicrometer-sized metallic tags (e.g., Nicewarner-Pena et al., Science 294:137-141, 2001). Nicewamer-Pena et al. (2001) disclose methods of preparing multimetal microrods encoded with submicrometer stripes, comprised of different types of metal. This system allows for the production of a very large number of distinguishable tags—up to 4160 using two types of metal and as many as 8×10⁵ with three different types of metal. Such tags may be attached to barcodes and detected. Methods of attaching metal particles, such as gold or silver, to oligonucleotides and other types of molecules are known in the art (e.g., U.S. Pat. No. 5,472,881).

Fullerenes Tags

Fullerenes may also be used as barcode tags. Methods of producing fullerenes are known (e.g., U.S. Pat. No. 6,358,375). Fullerenes may be derivatized and attached to other molecules by methods similar to those disclosed below for carbon nanotubes. Fullerene-tagged barcodes may be identified, for example, using various technologies.

Other types of known tags that may be attached to barcodes and detected are contemplated. Non-limiting examples of tags of potential use include quantum dots (e.g., Schoenfeld, et al., Proc. 7th Int. Conf. on Modulated Semiconductor Structures, Madrid, pp. 605-608, 1995; Zhao, et al., 1st Int. Conf. on Low Dimensional Structures and Devices, Singapore, pp. 467-471, 1995). Quantum dots and other types of tags may also be obtained from commercial sources (e.g., Quantum Dot Corp., Hayward, Calif.).

Carbon Nanotube Tags

Carbon nanotubes, such as single-walled carbon nanotubes (SWNTs), may also be used as tags. Nanotubes may be detected, for example, by Raman spectroscopy (e.g., Freitag et al., Phys. Rev. B 62:R2307-R2310, 2000). The characteristics of carbon nanotubes, such as electrical or optical properties, depend at least in part on the size of the nanotube.

Carbon nanotubes may be made by a variety of techniques known in the art, including but not limited to carbon-arc discharge, chemical vapor deposition via catalytic pyrolysis of hydrocarbons, plasma assisted chemical vapor deposition, laser ablation of a catalytic metal-containing graphite target, or condensed-phase electrolysis. (See, e.g., U.S. Pat. Nos. 6,258,401, 6,283,812 and 6,297,592.) Compositions comprising mixtures of different length carbon nanotubes may be separated into discrete size classes according to nanotube length and diameter, using any method known in the art. For example, nanotubes may be size sorted by mass spectrometry (See, Parker et al., “High yield synthesis, separation and mass spectrometric characterization of fullerene C60-C266,” J. Am. Chem. Soc. 113:7499-7503, 1991). Carbon nanotubes may also be purchased from commercial sources, such as CarboLex (Lexington, Ky.), NanoLab (Watertown, Mass.), Materials and Electrochemical Research (Tucson, Ariz.) or Carbon Nano Technologies Inc. (Houston, Tex.).

Carbon nanotubes may be derivatized with reactive groups to facilitate attachment to barcodes. For example, nanotubes may be derivatized to contain carboxylic acid groups (U.S. Pat. No. 6,187,823) that may be linked to amines using carbodiimide cross-linkers.

Nucleotide Tags

Nucleotides or bases, for example adenine, guanine, cytosine, or thymine may be used to tag molecular barcodes other than oligonucleotides and nucleic acids. For example, peptide based molecular barcodes may be tagged with nucleotides or purine or pyrimidines bases. Other types of purines or pyrimidines or analogs thereof, such as uracil, inosine, 2,6-diaminopurine, 5-fluoro-deoxycytosine, 7 deaza-deoxyadenine or 7-deaza-deoxyguanine may also be used as tags. Other tags include base analogs. A base is a nitrogen-containing ring structure without the sugar or the phosphate. Such tags may be detected by optical techniques, such as Raman or fluorescence spectroscopy. Use of nucleotide or nucleotide analog tags may not be appropriate where the target molecule to be detected is a nucleic acid or oligonucleotide, since the tag portion of the barcode may potentially hybridize to a different target molecule than the probe portion.

Amino Acid Tags

Amino acids may also be used to as tags. Amino acids of potential use as tags include but are not limited phenylalanine, tyrosine, tryptophan, histidine, arginine, cysteine, and methionine,

Cross-Linkers

Bifunctional cross-linking reagents may be used for various purposes, such as attaching tags to barcodes. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied (U.S. Pat. Nos. 5,603,872 and 5,401,511). Cross-linking reagents of potential use include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

Barcode Detection

Barcodes may be detected using any modality known in the art. For example, fluorescence spectroscopy may be used to detect a barcode. Several fluorescent dyes may be attached to a single barcode. The amount of dyes and the chemical properties of the dyes in a barcode will determine the fluorescence emission profile of the barcode. For a given barcode composition, signals may also be affected by relative distances between tags due to possible resonance energy transfers.

In other embodiments, Raman spectroscopy may be used to detect a barcode. Various Raman tags may be attached to a barcode for detection by known Raman spectroscopy techniques, such as SERS (surface enhanced Raman spectroscopy). In addition to attached Raman tags, the barcode backbone itself may be used as a Raman tag. Different base compositions of a DNA molecule produce different Raman signals that may be used as to identify a DNA-based barcode. Various specific detection modalities are discussed below.

Raman Spectroscopy

Surfaces for Raman Spectroscopy

Various modalities of Raman spectroscopy utilize enhancement of the Raman signal by proximity of the tagged (barcode) molecule to a surface. In certain modalites, such as surface enhanced Raman spectroscopy (SERS) or surface enhanced resonance Raman spectroscopy (SERRS), proximity to a Raman active metal surface, such as gold, silver, aluminum, platinum, copper or other metals, can enhance the Raman signal by up to six or seven orders of magnitude. Other types of compounds may also be used to enhance the signal in SERS, such as LiF, NaF, KF, LiCl, NaCl, KCl, LiBr, NaBr, KBr, LiI, NaI and KI. In particular, LiCl has been demonstrated to increase the relative signal of intensity of specific analytes (e.g. dAMP, deoxyadenosine, adenosine and adenine) between 2 and 100 fold. LiCl increases the relative intensity over 2 fold compared to the commonly used NaCl, depending on the analyte of interest. In other embodiments, NaBr or NaI may be better than LiCl for an analyte such as deoxyguanosine-monophosphate (dGMP).

Raman Detectors

Various methods of Raman detection are known in the art. One example of a Raman detection unit of use is disclosed in U.S. Pat. No. 6,002,471. As disclosed, the excitation beam is generated by either a Nd:YAG laser at 532 nm wavelength or a Ti:sapphire laser at 365 nm wavelength. Pulsed laser beams or continuous laser beams may be used. The excitation beam passes through confocal optics and a microscope objective, and is focused onto a target area. The Raman emission light from the Raman labels is collected by the microscope objective and the confocal optics and is coupled to a monochromonator for spectral dissociation. The confocal optics includes a combination of dichroic filters, barrier filters, confocal pinholes, lenses, and mirrors for reducing the background signal. Standard full field optics can be used as well as confocal optics. The signal may be detected by any known Raman detector.

Alternative examples of detection units are disclosed, for example, in U.S. Pat. No. 5,306,403, including a Spex Model 1403 double-grating spectrophotometer equipped with a gallium-arsenide (GaAs) photomultiplier tube (RCA Model C31034 or Burle Industries Model C3103402) operated in the single-photon counting mode.

Another exemplary Raman detection unit comprises a laser and Raman detector. The excitation beam is generated by a titanium:sapphire laser (Tsunami by Spectra-Physics) at a near-infrared wavelength (750˜950 nm) or a galium aluminum arsenide diode laser (PI-ECL series by Process Instruments) at 785 nm or 830 nm. Pulsed laser beams or continuous beams can be used. The excitation beam is reflected by a dichroic mirror (holographic notch filter by Kaiser Optical or an interference filter by Chroma or Omega Optical) into a collinear geometry with the collected beam. The reflected beam passes a microscope objective (Nikon LU series), and is focused onto an area where barcode-bound targets are located. The Raman scattered light is collected by the same microscope objective, and passes the dichroic mirror to the Raman detector. The Raman detector comprises a focusing lens, a spectrograph, and an array detector. The focusing lens focuses the Raman scattered light through the entrance slit of the spectrograph. The spectrograph (RoperScientific) comprises a grating that disperses the light by its wavelength. The dispersed light is imaged onto an array detector (back-illuminated deep-depletion CCD camera by RoperScientific). The array detector is connected to a controller circuit, which is connected to a computer for data transfer and control of the detector function.

Alternative excitation sources include a nitrogen laser (Laser Science Inc.) and a helium-cadmium laser (Liconox) (U.S. Pat. No. 6,174,677). The excitation beam may be spectrally purified with a bandpass filter (Corion) and may be focused using a 6× objective lens (Newport, Model L6X). The objective lens may be used to both excite the molecule of interest and to collect the Raman signal (Kaiser Optical Systems, Inc., Model HB 647-26N18. A holographic notch filter (Kaiser Optical Systems, Inc.) may be used to reduce Rayleigh scattered radiation. Other types of detectors may be used, such as charged injection devices, photodiode arrays or phototransistor arrays.

Alternative detection systems with respect to multiplex barcodes might include deciphering the difference in overlapping barcodes. One method to differentiate these barcodes may be standard DSP (digital signal processing) method so that, for example, the distance between different barcode elements in signal units (wavelength absorbance or shift from excitation, physical distance, tunneling conductivities, etc.) could be distinguished.

Any suitable form or configuration of Raman spectroscopy or related techniques known in the art may be used, for example normal Raman scattering, resonance Raman scattering, SERS, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

Micro-Electro-Mechanical Systems (MEMS)

Apparatus for barcode preparation, use and/or detection may be incorporated into a larger apparatus and/or system. In certain embodiments, the apparatus may comprise a micro-electro-mechanical system (MEMS). MEMS are integrated systems including mechanical elements, sensors, actuators, and electronics. All of those components may be manufactured by microfabrication techniques on a common chip, of a silicon-based or equivalent substrate (e.g., Voldman et al., Ann. Rev. Biomed. Eng. 1:401-425, 1999). The sensor components of MEMS may be used to measure mechanical, thermal, biological, chemical, optical and/or magnetic phenomena to detect barcodes. The electronics may process the information from the sensors and control actuator components such pumps, valves, heaters, etc. thereby controlling the function of the MEMS.

The electronic components of MEMS may be fabricated using integrated circuit (IC) processes (e.g., CMOS or Bipolar processes). They may be patterned using photolithographic and etching methods for computer chip manufacture. The micromechanical components may be fabricated using compatible “micromachining” processes that selectively etch away parts of the silicon wafer or add new structural layers to form the mechanical and/or electromechanical components.

Basic techniques in MEMS manufacture include depositing thin films of material on a substrate, applying a patterned mask on top of the films by some lithographic methods, and selectively etching the films. A thin film may be in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting. Methods for manufacture of nanoelectromechanical systems may also be used (See, e.g., Craighead, Science 290:1532-36, 2000.)

In some embodiments, apparatus and/or detectors may be connected to various fluid filled compartments, for example microfluidic channels or nanochannels. These and other components of the apparatus may be formed as a single unit, for example in the form of a chip (eg. semiconductor chips) and/or microcapillary or microfluidic chips. Alternatively, individual components may be separately fabricated and attached together. Any materials known for use in such chips may be used in the disclosed apparatus, for example silicon, silicon dioxide, polydimethyl siloxane (PDMS), polymethylmethacrylate (PMMA), plastic, glass, quartz, etc.

Techniques for batch fabrication of chips are well known in computer chip manufacture and/or microcapillary chip manufacture. Such chips may be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding, dry etching of silicon dioxide; and electron beam lithography. Methods for manufacture of nanoelectromechanical systems may be used for certain embodiments. (See, e.g., Craighead, Science 290:1532-36, 2000.) Various forms of microfabricated chips are commercially available from, e.g., Caliper Technologies Inc. (Mountain View, Calif.) and ACLARA BioSciences Inc. (Mountain View, Calif.).

In certain embodiments, part or all of the apparatus may be selected to be transparent to electromagnetic radiation at the excitation and emission frequencies used for barcode detection by, for example, Raman spectroscopy. Suitable components may be fabricated from materials such as glass, silicon, quartz or any other optically clear material. For fluid-filled compartments that may be exposed to various analytes, for example, nucleic acids, proteins and the like, the surfaces exposed to such molecules may be modified by coating, for example to transform a surface from a hydrophobic to a hydrophilic surface and/or to decrease adsorption of molecules to a surface. Surface modification of common chip materials such as glass, silicon, quartz and/or PDMS is known (e.g., U.S. Pat. No. 6,263,286). Such modifications may include, for example, coating with commercially available capillary coatings (Supelco, Bellafonte, Pa.), silanes with various functional (eg. polyethyleneoxide or acrylamide, etc).

In certain embodiments, such MEMS apparatus may be use to prepare molecular barcodes, to separate formed molecular barcodes from unincorporated components, to expose molecular barcodes to targets, and/or to detect molecular barcodes bound to targets.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Raman Detection of Molecular Barcodes

FIG. 3 illustrates exemplary single-stranded barcodes with attached Raman tags. The exemplary oligonucleotide sequences 301, 302, 303, 304 were synthesized by standard phosphoramidite chemistry. Tags for optical detection were attached to the oligonucleotides, including the fluorescent dyes ROX (carboxy-X-rhodamine) 310; FAM (6-carboxyfluorescine) 320; and TAMRA (tetramethylrhodamine) 330. The locations and identities of dye tags attached to each barcode are as indicated in FIG. 3. An amine group was attached to the 5′ end of three of the oligonucleotides 302, 303, 304 during synthesis.

### Example 2

### Raman Spectra of Molecular barcodes

The molecular barcodes shown in FIG. 3 were subjected to SERS. The SERS emission spectra are shown in FIG. 4. Samples containing 220 μl of a 1 μM solution of the indicated barcodes 301, 302, 303, 304 in the presence of silver colloids and LiCl were exposed to a laser beam for 100 ms and the surface enhanced Raman spectrum was recorded. Spectra were offset by about 1000 CCD count units. As shown in FIG. 4, each of the four molecular barcodes 301, 302, 303, 304 produced a distinguishable Raman emission spectrum, even though three of the molecular barcodes 302, 303, 304 contained the same Raman tag 330 attached to different locations on the same oligonucleotide sequence 302, 303, 304. This demonstrates the feasibility of producing distinguishable molecular barcodes using the disclosed methods.

### Example 3

The SERS spectra 801 802 803 804 805 806 generated by several exemplary Raman tags attached to nucleotides are shown in FIG. 8. The spectral pattern 801 802 803 804 805 806 produced from each Raman tag is readily distinguishable. Samples containing 220 μl of 1 μM barcode solution in the presence of silver colloids and LiCl were exposed to a laser beam for 100 ms and the surface enhanced Raman spectrum was recorded. The SERS emission spectra are shown for polyT[NeBu]T 801; polyT[EthdA]T 802; poly T[8Br-dA]T 803; poly T[2AmPur]T 804; [ThiSS] poly TdA 805 and [5Acrd]polydG [AmC7] 806.

### Example 4

One exemplary embodiment of the invention is illustrated. A nucleic acid sequence may be determined by using a decoding method, as illustrated in FIG. 5 and FIG. 6/7. A code component library or libraries (FIG. 6601 602 603 604) may be created such that each component of the library has an associated label (eg. Raman tag) that specifically and uniquely identifies the component (eg. a 3-mer). The nucleic acid is incubated with a component library or libraries to allow hybridization of the probes to the target sequence 605. The hybridized nucleic acids are manipulated through a micro-fluidic channel where they flow past an excitation source and a detector. Emission spectra of the code components may be detected and relayed to a data processing system. The sequence of the nucleic acid is determined by comparing the emission spectra and the order in which the emission spectra is detected to a database of spectra for code components associated with the label.

For example, a tissue sample may be obtained from a subject suspected of a disease (eg. by biopsy sample or possibly a blood sample). A single cell suspension may be generated by techniques known in the art and the cells lysed by one of several membrane disruption buffers to release the contents of the cells. Nucleic acids are isolated by methods known in the art (eg. phenol/chloroform extractions, gel purification etc.). The purified nucleic acid molecule is immobilized by attachment to a nylon membrane, 96-well microtiter plate or other immobilization substrate. The code components may be introduced, for example, one at a time or several at a time to the immobilized nucleic acid and allowed to interact with the molecule in a buffer of predetermined stringency (NaCl content). The coded probes are allowed to hybridize to a target nucleic acid. After hybridization of the first one or more code components, additional coded components may be added. Unhybridized code components and code components hybridized to each other are removed by extensive washing, leaving only code components that are hybridized to the immobilized nucleic acid. The code components are then sequentially removed and read by decoding the nucleic acid sequence that matches the code component. All or part of the sequence may be determined depending on the desired end point. This information may be compared to information known about a disease being tested and the presence or absence of particular sequences may determine the condition of the subject with respect to the disease in question. In one example, SNPs (single nucleotide polymorphisms) may be identified that correlate with a disease thus complete sequencing of an immobilized nucleic acid is unnecessary.

Alternatively, one or more code components may be immobilized on a surface such as a 96-well plate and these may be used to capture the corresponding nucleic acid molecule containing the target sequence such as a known SNP, insert or deletion that is a marker for a specific genotype etc. Rapid identification of a target sequence may be possible due to the sensitivity of the tag such as a Raman label.

### Example 5

One exemplary embodiment of the invention is illustrated. A protein or peptide (eg. a rare regulatory protein etc) may be purified as discussed previously. The purified protein/peptide is then used to generate antibodies (monoclonal antibodies may also be generated by techniques known in the art) by techniques well known in the art (Antibodies: A Laboratory Manual, Cold Spring Harbor Laboratory, Cold Spring Harbor Press, Cold Spring Harbor, N.Y., 1988). The reactivity of the antigen may be increased by co-administering adjuvants, such as Freund's complete or incomplete adjuvant. Antigenicity may be increased by attaching the antigen to a carrier, such as bovine serum albumin or keyhole limpet hemocyanin. The immune response of the animal may be increased by periodically administering a booster injection of the antigen. Antibodies 1206 are secreted into the circulation of the animal and may be obtained by bleeding or cardiac puncture. Antibodies 1206 may be separated from other blood components by well-known methods, such as blood clotting, centrifugation, filtration and/or immunoaffinity purification (e.g., using anti-rabbit antibodies) or affinity chromatography (e.g., Protein-A Sepharose column chromatography). These antibodies may then be linked (eg. covalently) to any one of the polymeric Raman labels illustrated in FIG. 12. The polymeric Raman labeled antibody may then be used to identify the protein out of an extract of many molecules. Alternatively, the polymeric Raman labeled antibody may be used to isolate several of the same proteins out of an extract of molecules for identification purposes, for further study of the protein of interest, to block the activity of the protein, identify a protein associated with a disease etc. Because the polymeric Raman labeled molecule (eg. polymeric Raman labeled Ab) is easily detected, it may also be used for diagnostic purposes to access the existence and or extent of a disease.

### Example 6

### Nucleic Acid Sequence Identification Using a Technique Illustrated in FIG. 5-7:

In one embodiment, nucleic acids may be modified using one or more Raman tags. Many small and unique Raman tags are available. In one example several Adenine analogs are illustrated in FIG. 13 that have strong and unique Raman signatures (others are illustrated in FIG. 8). In one example Raman tags may be linked to a nucleic acid through one or more base modifications and then these modified bases may be used to make phosphoramidites for chemical synthesis of oligonucleotides. Phosphoramidites of modified bases can be made by techniques known in the art (McBride, L. J. and Caruthers, M. H. (1983) “An investigation of several deoxynucleoside phosphoramidites useful for synthesizing deoxyoligonucleotides.” Tetrahedron Lett. 24:245-248).

In one embodiment, a code component may consist of a length of around 10-20 bases. For a 10-mer, this would be 4\hat{ }10 possible sequences and for 20-mer this would be 4\hat{ }20 possible sequences. In a practical application, the target sequence(s) is known or the sequences may be divided into a subset of sequences. Thus, an oligonucleotide may be for example labeled and identified by 1 or more Raman tags. In one example, if 10 different phosphoramidites may be used (each with a different Raman tag); 10 different oligos may be synthesized if there is 1 Raman tag per oligonucleotide sequence synthesized; 55 oligonucleotides may be synthesized if there are 2 Raman tags per oligonucleotide synthesized and 175 oligos may be synthesized if there are 3 tags per oligonucleotide. For example, phosphoramidites for oligonuleotide (code component) synthesis maybe used and these methods are known in the art. In one example, one component may be ATGCGACGT (SEQ ID NO:3) with kinetin (KN) as a tag (FIG. 13) and another may be GCTATAGCCG (SEQ ID NO:4) with Benzoyl-Adenine (BA) (FIG. 13) as a tag. Many of the barcode components may be pre-made and stored for later use.

In one embodiment, a barcode may be prepared by the following method. A barcode may be assembled from several code components. A barcode template may be a relatively long polynucleotide, for example, a DNA fragment of 40 nucleotides that may be synthesized by standard phosphoramidite chemistry:

(SEQ ID NO:5)

5′ ACGTCGCATT-CGGCTATAGC-TTTCTATAGCGCTATGGTAC 3′

The underlined section in this example may be the container section and the other sequence may be the probe section. Barcode components 5′-ATGCGACGT(KN)-3′ (SEQ ID NO:3) and 5′-GCTATAGCCG (BA)-3′ (SEQ ID NO:4) may be hybridized to the container section under standard conditions (for example, oligonucleotide concentrations in 1 to 10 μM in the presence of 200 mM NaCl, 10 mM Tris HCl, pH 7.5 and 1 mM EDTA). Therefore, in this example the probe section is represented by a 2-barcode component and its Raman signature is determined by both Kinetin and Benzoyl-Adenine as the Raman tags. To synthesize a different barcode template, the probe section and the container section are changed correspondingly; different barcode components (pre made) may be hybridized together to form a new barcode.

This technique may be used for example, to detect infectious agent by analyzing the presence of a DNA or RNA that correspond to the infectious agent. After collecting samples and extracting nucleic acids from the samples by techniques known in the art, the nucleic acids may be digested (eg. by restriction enzymes, limited DNase digestion, etc), and end-labeled with biotin by Terminal Transferase (available from New England Biolabs) in the presence of biotinylated-ddNTP (Perkin Elmer Life Sciences). After removing free nucleotides by gel filtration columns (Amersham-Pharmacia Biotech), the biotinylated DNAs may be captured on streptavidin-coated magnetic beads (Roche). The nucleic acids are then denatured with 0.1N NaOH (for DNA) to separate the 2 complementary strands. After neutralizing the target molecules, barcode molecules may be introduced in order to bind complementary sequences. One example of a binding/washing buffer may be 200 mM NaCl, 10 mM Tris HCl, pH 7.5 and 1 mM EDTA. A magnet (Dynal Corp) may be used for particle manipulation by methods known in the art.

In one example, the probe section of a barcode is complementary to a target sequence, for example, 5′ GTACCATAGCGCTATAGAAA 3′ (SEQ ID NO:6) barcode molecules will bind to the target sequences and thus be retained by a magnet in this example (Dyna beads, Dynal). The beads may be mixed with silver colloid (prepared from 1 mM AgNO3, diluted 1:2 with water), and 0.1 M LiCl (final concentration). When the particles pass through a Raman detector, the Raman signals (KN and BA) specifically associated with the barcode molecules may thus be detected. In this example the information may be used to confirm the presence or absence of a particular infectious agent in one or more samples.

### Example 7

Barcode-Antibody for Protein Detection

Another embodiment, may include preparation of Raman tagged barcode(s) as in example 6 but the barcode is then attached to an antibody for antigen detection (eg. a protein). Therefore, barcode preparations are generated and a DNA-tagged antibody may be made. For example, IgG antibody (eg. 200 μg (1.33 nmoles)) may be activated with 20 μg (52 nmoles) of sulfo-GMBS (Pierce Cat. No. 22324) in 200 μl of 0.1×PBS (diluted from 10×PBS, available from Ambion), for 30 min at 37° C. and then 30 min at room temperature. The solution is then passed though a PD-10 column (Amersham-Pharmacia) and the antibody-containing fractions are collected. Thiol modified DNA oligos may be synthesized by a commercial vendor (Qiagen-Operon). After reducing the disulfide bond (eg. DTT treatment) following instruction from the vendor, a DNA oligo (eg. 13 nmoles) may be mixed with a purified and activated antibody. The reaction is allowed to proceed for 2 hours at room temperature and 4° C. overnight. The DNA-antibody may then be purified by an ion exchange column (Amersham-Pharmacia) using for example a 0-2M NaCl gradient. The fractions containing both DNA and protein are collected. The sample is ready for antigen binding (protein detection) after desalting and concentration treatments using techniques known in the art.

Several methods may be used to immobilize antigens (eg. proteins) on solid supports. Preferably, for Raman detection, captured antibodies (capture antibody and detection antibody should recognize the same antigen, available from a commercial vendor, such as R&D System and BD Biosciences) may be immobilized on a gold surface by EDC chemistry (Benson et al, Science, 193, (2001), 1641-1644). The sample containing target antigens (eg. proteins) may be diluted in 1×PBS and then applied to the solid surface for specific binding. For example a DNA-tagged antibody is allowed to bind to a captured antigen (eg. protein target). Then a standard immunoassay procedure may be followed, typically, using 1×PBS and 0.05% Tween-20. Once a binding event occurs a complementary Raman-tagged DNA may be allowed to bind to the immobilized DNA oligos attached to the detection antibodies. Typically, a barcode molecules may be in a 10 nM concentration in 2×PBS and 1 g/ml yeast tRNA (Sigma). After washing with 1×PBS, silver colloid (prepared from 1 mM AgNO3, diluted 1:2 with water) may be added to the binding surface, LiCl is then added to 0.1M, followed by Raman measurement. Since the DNA oligos that are attached to an antibody are complementary to the probe section of a barcode, the presence of a barcode signature will indicate the presence of the antibody and thus the target antigen (protein). Several different antigens may be detected simultaneously by this method when different captured antibodies and DNA tagged detection antibodies are used in the same system.

All of the METHODS, COMPOSITIONS and APPARATUS disclosed and claimed herein can be made and used without undue experimentation in light of the present disclosure. It will be apparent to those of skill in the art that variations may be applied to the METHODS, COMPOSITIONS and APPARATUS described herein without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the claimed subject matter.

1. A method comprising:
obtaining a barcode comprising two or more tags attached to an organic molecule backbone;
binding the barcode to a target; and
detecting the barcode bound to the target.

2. The method of claim 1, wherein the backbone comprises at least one molecule selected from the group consisting of a nucleic acid, a peptide, a polysaccharide, a bio-polymer and a synthetic polymer.

3. The method of claim 2, wherein the nucleic acid is single-stranded DNA.

4. The method of claim 2, wherein the backbone comprises a nucleic acid covalently linked to a peptide.

5. The method of claim 1, wherein the tag is selected from the group consisting of nucleic acids, nucleotides, nucleotide analogs, base analogs, fluorescent dyes, peptides, amino acids, modified amino acids, organic moieties, Raman tags, quantum dots, carbon nanotubes, fullerenes, submicrometer metal particles, electron dense particles and crystalline particles.

6. The method of claim 1, wherein the backbone comprises one or more branched nucleic acids.

7. The method of claim 6, wherein the branches are located at predetermined sites along the backbone.

8. The method of claim 1, wherein the barcode is detected by a technique selected from the group consisting of fluorescent spectroscopy, Raman spectroscopy, Fourier transform infrared spectroscopy (FTIR), and surface plasmon resonance.

9. The method of claim 1, wherein the barcode binds to the target via a probe moiety.

10. The method of claim 1, wherein distinguishable barcodes are generated by attachment of the same tag to different sites along the same backbone.

11. The method of claim 1, wherein the target is selected from the group consisting of a protein, a peptide, a glycoprotein, a lipoprotein, a prion, a nucleic acid, a polynucleotide, an oligonucleotide, a lipid, a fatty acid, a carbohydrate, a glycolipid, a phospholipid, a sphingolipid, a lipopolysaccharide, a polysaccharide, a eukaryotic cell, a prokaryotic cell, a bacterium, a phage, a virus and a pathogen.

12. A method comprising:
obtaining a nucleic acid template comprising a container section and a probe section; and
hybridizing one or more tagged oligonucleotides to the container section to create a barcode.

13. The method of claim 12, further comprising binding the barcode to a target.

14. The method of claim 13, further comprising detecting the barcode bound to the target.

15. A method for making a polymeric Raman label comprising:
obtaining two or more monomeric units; and
polymerizing the monomeric units to make a polymeric Raman label.

16. The method of claim 15, wherein each monomeric unit comprises a Raman tag.

17. The method of claim 16, wherein each Raman tag in a single polymeric Raman label is different.

18. The method of claim 16, wherein the Raman tags are attached to the backbone of the polymeric Raman label through spacer moieties.

19. The method of claim 15, wherein Raman tags are attached to the polymeric Raman label after polymerization of the monomeric units.

20. The method of claim 15, further comprising deprotecting a functional group at one end of a monomeric unit and forming a covalent bond between the monomeric unit and the polymeric Raman label.

21. The method of claim 15, further comprising generating subpolymeric units, each subpolymeric unit comprising a predetermined number of monomeric units.

22. The method of claim 15, further comprising attaching the polymeric Raman label to a solid support.

23. The method of claim 22, wherein the solid support is a nanoparticle or bead.

24. The method of claim 15, further comprising attaching a probe to the polymeric Raman label.

25. The method of claim 24, further comprising binding the probe to a target.

26. The method of claim 25, further comprising detecting the probe bound to the target.

27. A polymeric Raman label comprising:
two or more monomeric units covalently attached together;
two or more Raman tags; and
at least one probe.

28. The polymeric Raman label of claim 27, further comprising a nanoparticle or bead attached to the polymeric Raman label.

29. The polymeric Raman label of claim 27, wherein each Raman tag in the label is different.

30. The polymeric Raman label of claim 27, further comprising two or more copies of each Raman tag.

31. A system comprising:
an imaging instrument;
at least one barcode linked to a probe; and
at least one target bound to the probe.

32. The system of claim 31, wherein the imaging instrument is selected from the group consisting of a fluorescent instrument, a Raman instrument, and an FTIR instrument.

33. The system of claim 31, wherein each barcode comprises two or more Raman tags.

34. The system of claim 33, wherein each Raman tag in a single barcode has a different Raman emission spectrum.


SEQUENCE LISTING (6 sequences)

SEQ ID NO: 1 - DNA, 16 bp, Artificial
  Oligonucleotide
agaaagtacatatgtc

SEQ ID NO: 2 - DNA, 16 bp, Artificial
  Oligonucleotide
agtaagaacatatgtc

SEQ ID NO: 3 - DNA, 9 bp, Artificial
  Oligonucleotide
atgcgacgt

SEQ ID NO: 4 - DNA, 10 bp, Artificial
  Oligonucleotide
gctatagccg

SEQ ID NO: 5 - DNA, 40 bp, Artificial
  Oligonucleotide
acgtcgcattcggctatagctttctatagcgctatggtac

SEQ ID NO: 6 - DNA, 20 bp, Artificial
  Oligonucleotide
gtaccatagcgctatagaaa